SEQUENCE LISTING (33 sequences)

SEQ ID NO: 1 - DNA, 12 bp, Artificial Sequence
  May be a sequence in a naturally-occurring nucleic acid
acgttttgacat

SEQ ID NO: 2 - DNA, 11 bp, Artificial Sequence
  May be a sequence in a naturally-occurring nucleic acid
acgttttacat

SEQ ID NO: 3 - DNA, 12 bp, Artificial Sequence
  May be a sequence in a naturally-occurring nucleic acid
acgttttgacat

SEQ ID NO: 4 - DNA, 12 bp, Artificial Sequence
  May be a sequence in a naturally-occurring nucleic acid
acgttttcacat

SEQ ID NO: 5 - DNA, 12 bp, Artificial Sequence
  May be a sequence in a naturally-occurring nucleic acid
acgttttgacat

SEQ ID NO: 6 - DNA, 12 bp, Artificial Sequence
  May be a sequence in a naturally-occurring nucleic acid
acgttttcacat

SEQ ID NO: 7 - DNA, 12 bp, Artificial Sequence
  May be a sequence in a naturally-occurring nucleic acid
acgttttcacat

SEQ ID NO: 8 - DNA, 12 bp, Artificial Sequence
  May be a sequence in a naturally-occurring nucleic acid
acgttttcacat

SEQ ID NO: 9 - DNA, 12 bp, Artificial Sequence
  May be a sequence in a naturally-occurring nucleic acid
acgtttttacat

SEQ ID NO: 10 - DNA, 12 bp, Artificial Sequence
  May be a sequence in a naturally-occurring nucleic acid
acgttttcacat

SEQ ID NO: 11 - DNA, 12 bp, Artificial Sequence
  May be a sequence in a naturally-occurring nucleic acid
acgttttcacat

SEQ ID NO: 12 - DNA, 12 bp, Artificial Sequence
  May be a sequence in a naturally-occurring nucleic acid
acgttttcacat

SEQ ID NO: 13 - DNA, 12 bp, Artificial Sequence
  May be a sequence in a naturally-occurring nucleic acid
acgttttcacat

SEQ ID NO: 14 - DNA, 12 bp, Artificial Sequence
  May be a sequence in a naturally-occurring nucleic acid
acgttttcacat

SEQ ID NO: 15 - DNA, 12 bp, Artificial Sequence
  May be a sequence in a naturally-occurring nucleic acid
acgttttcacat

SEQ ID NO: 16 - DNA, 12 bp, Artificial Sequence
  May be a sequence in a naturally-occurring nucleic acid
acgttttcacat

SEQ ID NO: 17 - DNA, 12 bp, Artificial Sequence
  May be a sequence in a naturally-occurring nucleic acid
acgttttgacat

SEQ ID NO: 18 - DNA, 12 bp, Artificial Sequence
  May be a sequence in a naturally-occurring nucleic acid
acgttttcacat

SEQ ID NO: 19 - DNA, 12 bp, Artificial Sequence
  May be a sequence in a naturally-occurring nucleic acid
acgttttgacat

SEQ ID NO: 20 - DNA, 12 bp, Artificial Sequence
  May be a sequence in a naturally-occurring nucleic acid
acgttttcacat

SEQ ID NO: 21 - DNA, 12 bp, Artificial Sequence
  May be a sequence in a naturally-occurring nucleic acid
acgtttttacat

SEQ ID NO: 22 - DNA, 12 bp, Artificial Sequence
  May be a sequence in a naturally-occurring nucleic acid
acgttttgacat

SEQ ID NO: 23 - DNA, 12 bp, Artificial Sequence
  May be a sequence in a naturally-occurring nucleic acid
acgttttaacat

SEQ ID NO: 24 - DNA, 12 bp, Artificial Sequence
  May be a sequence in a naturally-occurring nucleic acid
acgttttgacat

SEQ ID NO: 25 - DNA, 12 bp, Artificial Sequence
  May be a sequence in a naturally-occurring nucleic acid
acgttttgacat

SEQ ID NO: 26 - DNA, 12 bp, Artificial Sequence
  May be a sequence in a naturally-occurring nucleic acid
acgttttgacat

SEQ ID NO: 27 - DNA, 12 bp, Artificial Sequence
  May be a sequence in a naturally-occurring nucleic acid
acgttttgacat

SEQ ID NO: 28 - DNA, 12 bp, Artificial Sequence
  May be a sequence in a naturally-occurring nucleic acid
acgttttgacat

SEQ ID NO: 29 - DNA, 15 bp, Artificial Sequence
  May be a sequence in a naturally-occurring nucleic acid
acgttttgtccacat

SEQ ID NO: 30 - DNA, 11 bp, Artificial Sequence
  May be a sequence in a naturally-occurring nucleic acid
acgttttacat

SEQ ID NO: 31 - DNA, 15 bp, Artificial Sequence
  May be a sequence in a naturally-occurring nucleic acid
acgaaaagtccacat

SEQ ID NO: 32 - DNA, 11 bp, Artificial Sequence
  May be a sequence in a naturally-occurring nucleic acid
acgttttacat

SEQ ID NO: 33 - DNA, 9 bp, Artificial Sequence
  May be a sequence in a naturally-occurring nucleic acid
ctgctgctg

(19) United States
(12) Patent Application Publication
(10) Pub. No.: US 2019/0005192 A1
(43) Pub. Date: Jan. 3, 2019

(54) Reliable and Secure Detection Techniques for Processing Genome Data in Next Generation Sequencing (NGS)

(71) Applicant: Bahram Ghaffarzadeh KERMANI, Los Altos, CA (US)

(72) Inventor: Bahram Ghaffarzadeh KERMANI, Los Altos, CA (US)

(21) Appl. No.: 15/897,152

(22) Filed: Feb. 14, 2018

Related U.S. Application Data
(60) Provisional application No. 62/458,997, filed on Feb. 14, 2017.
(60) Provisional application No. 62/458,788, filed on Feb. 14, 2017.
(60) Provisional application No. 62/458,720, filed on Feb. 14, 2017.
(60) Provisional application No. 62/515,174, filed on Jun. 5, 2017.
(60) Provisional application No. 62/576,075, filed on Oct. 23, 2017.

(51) Int. Cl. G06F 19/22; C12Q 1/6883; G06F 19/18; G16H 50/30; G16H 50/20

(52) U.S. Cl. CPC G06F 19/22; C12Q 1/6883; C12Q 2600/156; G16H 50/30; G16H 50/20; G06F 19/18

(57) ABSTRACT

Genetic samples are obtained from separate people, and at least a portion of each are purposefully combined before testing to form a pooled genetic sample. The pooled genetic sample is tested for the presence of a signature for a given known ailment. DNA identification uses discovered InDels in a region of InDel variation in a genetic sample. A pair-wise comparison is performed to reference InDels, and a distance is measured between the first InDel and the reference Indel. Reference kmers are identified in a reference genome, and in a test sample. The plurality of sample kmers are filtered to those which have a 1 edit distance from a corresponding one of the plurality of reference kmers. Reads that have kmers that do not have a 1 edit distance from the corresponding one of the plurality of reference kmers are identified, and multiple single-mutations are eliminated from candidate InDel reads.

The present application claims priority from U.S. Provisional No. 62/458,997 entitled “Multi-round Genome Processing Methods for NGS-based Genetic Tests”, filed Feb. 14, 2017; and also from U.S. Provisional No. 62/458,788 entitled Methods and Applications of High-fidelity Condition Detection using Genome Sequencing Techniques”, filed Feb. 14, 2017; and also from U.S. Provisional No. 62/458,720 entitled “Two-step Optimization of Analytical and Algorithmic Methods for High Accuracy Genomic Applications”, filed Feb. 14, 2017; and also from U.S. Provisional No. 62/515,174 entitled “DNA Sequencing Signatures for Early Detection of Cancer via Liquid Biopsy”, filed Jun. 5, 2017; and also from U.S. Provisional No. 62/576,075 entitled “Method and Apparatus for Enabling High-Accuracy Low-Cost Population-Level Genetic Testing”, filed Oct. 23, 2017, the entirety of all of which are expressly incorporated herein by reference.

### BACKGROUND OF THE INVENTION

### 1. Field of the Invention

The present invention relates to genomic testing and improved techniques and method for variant detection within a WGS (whole genome sequencing) or partial genome modality.

### 2. Background of Related Art

Genome sequencing determines the order of DNA nucleotides, or bases, in a genome, i.e., the order of As, Cs, Gs and Ts that make up an organism's DNA. The human genome is a sequence of over 3 billion of these genetic ‘letters’. Genetic testing identifies a variant of these genetic letters from a norm or reference genome to confirm or rule out a suspected genetic condition or determine a person's risk of developing a genetic disorder. The variant may be a single incorrect ‘letter’, or the variant may be an insertion or deletion of a segment of one or more pairs of ‘letters’ (InDel).

Conventional genetic testing currently has unique and significant challenges, both in reliability of detection of a given genetic condition, and also with respect to the resulting ethics and privacy concerns regarding a person's genetic information, including protection from genetic discrimination, e.g., by insurers, health care providers, etc.

Significant developments and discoveries continue to occur in the field of genetics, requiring new test methods and techniques with respect to genetic testing. A genetic test today may have a low reliability of accuracy resulting in an uncertain clinical utility, whereas a future genetic test or technique may have higher result accuracy leading to increased clinical utility.

Reliability in a genetic test is conventionally improved to a certain extent by increasing the number of sequences of a given sample, e.g., often >500×. However, increased sequencing comes at the expense of time required, and thus the overall cost therefore.

There is a need for methods and techniques of genetic testing and analysis that reduces the need for a high number of sequences (often >500×) and thus the cost and speed of testing of an individual sample. There is also a need for methods and techniques to better scale up to efficient testing of a larger number of DNA samples from a corresponding larger number of individuals.

Moreover, genetic testing for cancer susceptibility, genetic diseases (including rare diseases) has become an accepted part of oncologic care. Germline testing for inherited predisposition is well established as part of the care of individuals who may be at hereditary risk for cancers of the breast, ovary, colon, stomach, uterus, thyroid, and other currently known primary sites.

Germline cells are those that are each descended or developed from earlier cells in the series, regarded as continuing through successive generations of an organism. Somatic cells are diploid containing two copies of each chromosome, whereas the germline cells are haploid as they only contain one copy of each chromosome. Genes and chromosomes can mutate in either somatic or germinal tissue. Somatic mutations occur in a single body cell and cannot be inherited (only tissues derived from mutated cell are affected). Germline mutations occur in gametes—a mature germ cell that is able to unite with another of the opposite sex in sexual reproduction to form a zygote—and can be passed on to offspring (every cell in the entire organism will be affected.) The offspring may also have its own private de novo mutations. These mutations are not transmitted from either parent.

Germline genetic testing is distinct from somatic genetic profiling of cancer tissue to have diagnosis, predict prognosis or treatment response. Germline testing conventionally involves analysis of DNA from blood or saliva for inherited mutations in specific genes that are associated with the type of cancer (other genetic conditions or predispositions) seen in the individual or family seeking assessment. When identified, such high-penetrance mutations usually could lead to in a significant alteration in the function of the corresponding gene product and are associated with large increases in cancer risk.

Most inherited cancer susceptibility arises from a number of DNA sequence variants, each of which, in isolation, confers a limited increase in risk. The genomic locations of a number of these low-penetrance variants (LPVs) have been defined through genome-wide association studies (GWAS).

In genomic risk assessment, the variants associated with disease risk in an individual's genomic profile are identified (or genotyped) and translated into absolute risk estimates through the use of various algorithms and biological samples. There is currently great uncertainty whether conventional algorithms are well calibrated or whether the risk estimates conventionally provided through genomic risk assessment are accurate. There is a need for more reliably accurate genomic analysis techniques and algorithms.

Conventional germline tests for certain high-penetrance predispositions or mutations in appropriate populations have clinical utility, meaning that they inform clinical decision making and facilitate the prevention or amelioration of adverse health outcomes. However, conventional genetic tests for intermediate-penetrance mutations and genomic profiles of variants linked to LPVs (low-penetrance variants) are of uncertain clinical utility because the cancer risk associated with the mutation or variant is generally too small (or unreliably detected) to form an appropriate basis for clinical decision making. Clinically ambiguous test results could produce unjustified alarm and may lead patients to request unnecessary screening and other preventive care that can cause physical discomfort or harm and increase costs. On the other hand, false reassurance may result from ambiguous test results or results associated with minimal cancer risk discouraging individuals from taking appropriate preventive measures. There is a need for more accurate and useful genomic testing and profiling, and a need for protection of genetic privacy.

Conventional genetic testing has a low reliability of accurate detection of intermediate-penetrance mutations or low-penetrance mutations. Thus, there is a need for a more reliable test for intermediate-penetrance mutations and even for testing for low-penetrance mutations.

Conditions such as cancer are detected by sequencing a material that stems from a mixture of N+1 genomes: G0, G1, G2, . . . , Gn. G0 is often from the germline source, i.e., it originates from the normal (often healthy) cells. G1, G2, . . . , Gn come from N sources. Often, these N sources are ultimately derived from G0. An example of this is in the case of multi-clonal cancer, where each of Gi (i=1, 2, . . . , n) come from a certain tumor clone Ci (i=1, 2, . . . , n), respectively. The term “GiSet” as used herein represents the set of genomes G1, G2, . . . , Gn.

The GiSet is sorted based on density, and density is related to the size of the tumor, or the number of the elements (cell-free DNA/RNA or reads), etc. Thus, G0 is often larger (in terms of number of molecules and/or number of reads) than all the other genomes G1, G2, . . . , Gn. Often, G0 is much greater than even G1. Also, for instance in the case of a prominent tumor clone or a single tumor clone, G1 is much greater than G2, etc. Thus, in most scenarios there are only detectable levels of G0 and G1, and even then G0>>G1.

The main goals of genetic testing are (1) detection of the existence of an anomaly, or variant, and (2) characterization of the detected variant:

In particular, the first goal of genetic testing is the detection of the existence of any of the GiSet. Existence of any of G0, G1, etc. which would indicate the existence of a particular variant or disease. In other words, for the cancer example, it is not known if the person has or does not have cancer (detectable via sources like cell-free DNA). Early (and reliable) detection of a relevant disease such as cancer would be possible if a detection technique is able to detect the existence of any of the GiSet, even in a situation with only a small number of any of the GiSet. The earlier the progression of the relevant disease, the fewer of the GiSet will exist. On the other hand, detection techniques which require larger numbers of any of the GiSet results in a later or delayed detection of the relevant disease. Thus, there is a need for an improved detection technique which can result in earlier detection of disease.

Another goal of genetic testing is characterization of the detected GiSet by articulating all existing variations in the detected GiSet, or at least all existing variations in G1. Different variations of the detected GiSet often exist when at least one source of cancer (G1) or similar variant exists, and one needs to find the variants that are specific to the G1 constituent of the mixture.

Detection and characterization of the GiSet is conventionally achieved by:

1. Making a “reduced sample/genome” from an original sample/genome. This reduction is done by genome enrichment of the loci of interest (LOI) within the sample. The LOI often comprises a very small part of the genome, e.g., <1%. The enrichment step is done by either hybridization-based or amplicon-based methods such as PCR.

2. Optionally, a tag is added to the genome fragments to enable Molecular Barcoding. The tagging step can be performed either before or after Step 1.

3. A high coverage (often >500×) sequencing is conventionally required on the reduced sample to provide a reliable result, but as mentioned high coverage sequencing takes more time and thus increases costs.

4. Optionally, the tagged fragments are uniquified, to reduce the biases caused by the assay (in particular, the PCR step). As the coverage depth increases in Step 3, the usage of molecular barcoding becomes inevitable.

5. The reads are mapped to the reference genome.

6. Variants are called (i.e., identified) on the mapped reads.

Conventional genomic tests to sequence a complete or a partial genome modality suffer in that the genomic tests often do not have sufficient information content to successfully, or reliably, perform the task. An example of this is methylation (by bisulfite conversion). Another example is the mapping of very short reads to the reference genome.

Reliability of the genomic test's result may also be adversely affected by variations that exist in the normal DNA of the individual. For example, consider a mixture of genomes where G2 exists at a very low concentration in G1—where G1 is the normal genome. Also, assume G2 is actually derived from G1 (such as in cancer cells). Assume the purpose of the genomic test is to pick mutations (variants) that are unique to G2. Since both G1 and G2 are expected to carry the variations on G1, then there is a chance for false-positives, where a detected private mutation of G2 is actually from G1 which happened to have weak support.

In order to overcome some of the shortcomings of the single (affected sample only) tests, differential (affected vs normal sample) tests are conventionally performed. In this mode, both an affected sample and a normal sample undergo the same biochemistry, and thus in theory both experience the same biases. The results of the affected vs. normal tests are expected to work better than a test on the affected sample only. However, the inventors hereof have recognized that there are nevertheless problems with affected vs. normal tests.

For instance, both affected and normal samples should be available at the time of acquisition, but in practice this may not be possible, or may be expensive to achieve. For example, providing a sample from a healthy (normal) tissue may not be accessible, or may even cause ethical issues if the sample's volume is not negligible, or if the normal tissue is hard to access, etc. Thus, the inventor has appreciated that a variation in acquisition time of the affected vs normal sample may affect the results. The inventors have also appreciated that the modalities of the affected and normal samples must match. For instance, if the affected test is RNA-based, the normal sample must also be RNA-based. Also, the quantities of the affected and normal samples must match. If not, the differential mode of analysis would be biased, and even if similar volume samples are attempted, even just sampling error between the two can cause an imbalance in the acquired samples.

Furthermore, the inventor appreciated that the sample acquisition mode could be the same for both the affected and normal samples. For instance, if one is tissue-based, the other one should also be tissue-based and from the same tissue. The normal could also be obtained from the peripheral blood. However, if the source is limited, such as in tissue, the amount of material provided for the normal sample is also limited (similar to that of the affected sample). Even the fact that half of the information in an affected vs normal sample comes from the need to also analyze normal cells/samples, means that the cost of the test for the affected sample is thus actually doubled (if we discount the benefits of the normal sample).

Also, analysis of affected and normal samples uses differential information at the micro-level, for instance at the read level. As a result, any stochastic bias that would exist in the assay will then bias the results. For any subsequent test, another sample of the normal sample should be provided to pair with the affected sample. The read length for the normal sample is bound to be the same as that of the affected samples, which may reduce the information content as the affected samples may have a reduced length, e.g., those derived from a cell-free DNA source.

The general results of genetic tests and genomic risk profiles are conventionally available directly to consumers (DTC) or as laboratory developed tests (LDTs), usually through Internet portals. The DTC model allows individuals to submit to a genetic test and receive results directly from the company that provides the test, outside of an established provider-patient relationship. But the present inventor has concerns regarding the safety, effectiveness, and risks associated with DTC provision of the results of genetic tests of uncertain clinical utility, and of course there is a concern about genetic privacy.

Consumers who receive test results directly may have pursued testing without the benefit of pre- or post-test counseling and may be unprepared to receive ambiguous or clinically significant results from tests with established clinical utility. Where clinical utility is uncertain, providers face the added challenge of explaining why test results lack clinical consequences. There is also a concern that risk calculations for the same conditions derived from DNA samples from the same individual can conventionally yield disparate results when analyzed by different DTC laboratories.

With these concerns in mind, only limited genetic testing for disease susceptibility has typically been offered as LDTs or in some cases as directly to consumers when the individual being tested has a personal or family history suggestive of susceptibility to a given illness that has a known genetic marker capable of reliable detection. Individuals who order DTC (direct-to-consumer) tests of uncertain clinical utility may ask their health care providers for help interpreting test results and for access to follow-up care, but this poses significant challenges to the providers who had no role in initiating or recommending the uncertain genetic testing in the first place. There is a need for improved DTC techniques and methods.

### SUMMARY OF THE INVENTION

In accordance with a first aspect of the invention, a method of performing genetic testing comprises obtaining a first genetic sample from a first person, and obtaining a second genetic sample from a second person. At least a portion of the first genetic sample is purposefully mixed with at least a portion of the second genetic sample into a pooled genetic sample. The pooled genetic sample is tested for the presence of a signature for a given known ailment.

In accordance with another aspect of the invention, a method of performing DNA identification using discovered InDels, comprises identifying at least one region of InDel variation in a genetic sample. A low-coverage sequencing of the genome is performed, and presence of a first InDel is detected in a loci of the region of InDel variation. A pair-wise comparison of the first InDel to a reference InDel is performed, and a distance is measured between the first InDel and the reference Indel.

In accordance with yet another aspect of the invention, a method of identifying a read with an InDel mutation in a genetic test comprises identifying a plurality of reference kmers in a reference genome. A plurality of sample kmers is identified in a test sample. The plurality of sample kmers are filtered to those which have a 1 edit distance from a corresponding one of the plurality of reference kmers. Reads that have kmers that do not have a 1 edit distance from the corresponding one of the plurality of reference kmers are identified, and multiple single-mutations are eliminated from candidate InDel reads.

### BRIEF DESCRIPTION OF THE DRAWINGS

Features and advantages of the present invention will become apparent to those skilled in the art from the following description with reference to the drawings, in which:

FIG. 1 illustrates an exemplary next generation sequencing (NGS) genome processing system on which some and/or parts of the techniques and methods for multi-round genome processing described herein may be implemented.

FIG. 2 illustrates an example DNA sequencing system 200 on which some and/or parts of the techniques and methods for multi-round genome processing described herein may be implemented.

FIG. 3 shows a general testing process for genetic testing in accordance with the principles of the present invention.

FIG. 4 shows DNA identification based on discovered InDels in low-coverages reads, in accordance with a first embodiment.

FIG. 5 shows an alternate, more general method of DNA identification based on discovered InDels in low-coverages reads, in accordance with a second embodiment.

FIG. 6 shows genome testing with bias minimized or removed, in accordance with an embodiment of the present invention.

FIG. 7 shows identification of reads containing InDels, in accordance with an embodiment of the present invention.

FIG. 8 shows an alternative method of identifying the reads with potential InDels, including other mutations.

FIG. 9 shows testing of circulating tumor cells (CTCs) for early detection of cancer via liquid biopsy, in accordance with the principles of the present invention.

FIG. 10 shows a first exemplary method to contrast variant-identifying signals in a tumor sample, with signals in a normal, to cancel out the effects of the normal.

FIG. 11 shows a second exemplary method to contrast variant-identifying signals in a tumor sample, with signals in a normal, to cancel out the effects of the normal.

FIG. 12 shows a third exemplary method to contrast variant-identifying signals in a tumor sample, with signals in a normal, to cancel out the effects of the normal.

FIG. 13 shows a fourth exemplary method to contrast variant-identifying signals in a tumor sample, with signals in a normal, to cancel out the effects of the normal.

### DETAILED DESCRIPTION OF ILLUSTRATIVE EMBODIMENTS

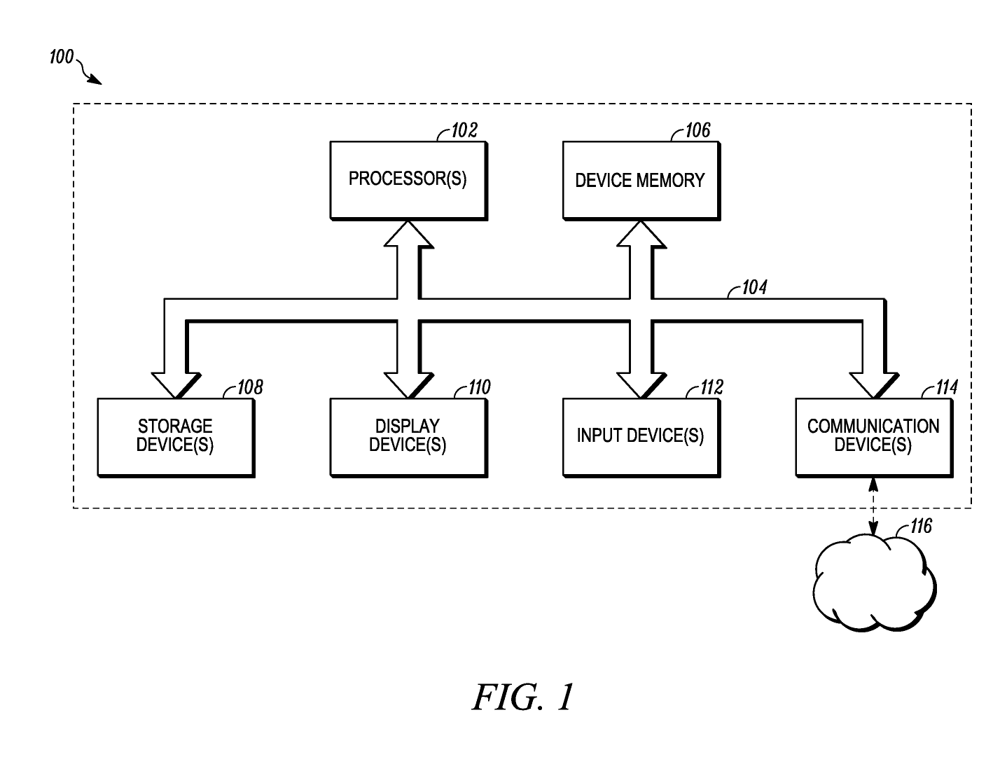

FIG. 1 illustrates an exemplary next generation sequencing (NGS) genome processing system on which some and/or parts of the techniques and methods for multi-round genome processing described herein may be implemented. Computer system 100 includes, but is not limited to, one or more processors 102 operationally coupled to memory 106 over one or more buses such as bus 104. Depending on specific implementations and form factors, computer system 100 may also include storage device(s) 108, display device(s) 110, input device(s) 112, and communication device(s) 114.

A processor 102 is a hardware device configured to execute sequences of instructions in order to perform various operations such as, for example, arithmetical, logical, and input/output operations. A typical example of a processor is a central processing unit (CPU), but it is noted that other types of processors such as vector processors and array processors can perform similar operations. Examples of hardware devices that can operate as processors include, but are not limited to, microprocessors, microcontrollers, digital signal processors (DSPs), systems-on-chip, and the like. Processor 102 is configured to receive executable instructions over one or more data and/or address buses such as bus 104. Bus 104 is configured to couple various device components, including memory 106, to processor(s) 102. Bus 104 may include one or more bus structures (e.g., such as a memory bus or memory controller, a peripheral bus, and a local bus) that may have any of a variety of bus architectures. Memory 106 is configured to store data and executable instructions for processor(s) 102. Memory 106 may include volatile and/or non-volatile memory such as read-only memory (ROM) and random-access memory (RAM). For example, a basic input/output system (BIOS) containing the basic executable instructions for transferring information between system components (e.g., during start-up) is typically stored in ROM. RAM typically stores data and executable instructions that are immediately accessible and/or being operated on by processor(s) 102 during execution. Memory 106 is an example of non-transitory computer-readable medium.

Computer-readable media may include any available medium that can be accessed by a computer system (and/or the processors thereof) and includes both volatile and non-volatile media and removable and non-removable media. One example of non-transitory computer-readable media is storage media. Storage media includes media implemented in any method or technology for storage of information such as computer-readable instructions, data structures, program modules, and/or other data. Examples of storage media include, but are not limited to, RAM, ROM, electrically erasable programmable read-only memory (EEPROM), removable memory such as flash memory and solid state drives (SSD), compact-disk read-only memory (CD-ROM), digital versatile disks (DVD) and other optical disks, magnetic cassettes, magnetic tapes, magnetic disks or other magnetic storage devices, electromagnetic disks, and any other medium which can be used to store the desired information and which can be accessed and read by a computer system. Another example of computer-readable media is communication media. Communication media typically embody computer-readable instructions, data structures, program modules, or other data, in a modulated data signal such as a carrier wave or other transport mechanism and includes any information delivery media. By way of example, and not limitation, communication media includes wired media such as a wired network or direct-wired connection, and wireless media such as acoustic, radio frequency (RF), infrared and other wireless media.

Computer system 100 may include, and/or have access to, various non-transitory computer-readable media that is embodied in one or more storage devices 108. Storage device(s) 108 may be coupled to processors(s) 102 over one or more buses such as bus 104. Storage device(s) 108 are configured to provide persistent storage of executable and other computer-readable instructions, data structures, program modules, and other data for computer system 100 and/or for its users. In various embodiments and form factors of computer system 100, storage device(s) 108 may include persistent storage media of one or more types including, but not limited to, electromagnetic disks (e.g., hard disks), optical storage disks (e.g., DVDs and CD-ROMs), magneto-optical storage disks, solid-state drives, flash memory cards, universal serial bus (USB) flash drives, and the like. By way of example, storage device(s) 108 may include a hard disk drive that stores the executable instructions of an Operating System (OS) for computer system 100, the executable instructions of one or more computer programs, clients, and other computer processes that can be executed on the computer system, and any OS and/or user data in various formats.

Computer system 100 may also include one or more display devices 110 and one or more input devices 112 that are coupled to processor(s) 102 over one or more buses such as bus 104. Display device(s) 110 may include any devices configured to receive information from, and/or present information to, user(s) of computer system 100. Examples of such display devices include, but are not limited to, cathode-ray tube (CRT) monitors, liquid crystal displays (LCDs), light emitting diode (LED) displays, field emission (FED, or “flat panel” CRT) displays, plasma displays, electro-luminescent displays, and any other types of display devices. Input device(s) 112 may include a general pointing device (e.g., such as a computer mouse, a trackpad, or an equivalent spatial-input device), an alphanumeric input device (e.g., such as a keyboard), and/or any other suitable human interface device (HID) that can communicate commands and other user-generated information to processor(s) 102.

Computer system 100 may include one or more communication devices 114 that are coupled to processor(s) 102 over one or more buses such as bus 104. Communication device(s) 114 are configured to receive and transmit data from and to other devices and computer systems. For example, communication device(s) 114 may include one or more USB controllers for communicating with USB peripheral devices, one or more network storage controllers for communicating with storage area network (SAN) devices and/or network-attached storage (NAS) devices, one or more network interface cards (NICs) for communicating over wired communication networks, and/or one or more wireless network cards for communicating over a variety of wireless data-transmission protocols such as, for example, IEEE 802.11 and/or Bluetooth. Using communication device(s) 114, computer system 100 may operate in a networked environment using logical and/or physical connections to one or more remote computer systems and/or other computing devices. For example, computer system 100 may be connected to one or more remote computers that provide access to block-level data storage over a SAN protocol and/or to file-level data storage over a NAS protocol. In another example, computer system 100 may be connected to one or more networks 116 over connections that support one or more networking protocols. Network(s) 116 may include, without limitation, a local area network (LAN), a wide area network (WAN), a global network (e.g., the Internet), and/or any other type of network or combination of networks.

Some embodiments and/or parts of the techniques for multi-round genome processing described herein may be implemented as a computer program product that may include sequences of instructions stored on non-transitory computer-readable media. These instructions may be used to program one or more computer systems that include one or more special-purpose or general-purpose processors (e.g., CPUs) or equivalents thereof (e.g., such as processing engines, processing cores, etc.). When executed by the processor(s), the sequences of instructions cause the computer system(s) to perform the operations according to some of the embodiments of the techniques described herein. Additionally, or instead of, some embodiments of the techniques described herein may be practiced in distributed computing environments that may involve more than one computer system. One example of a distributed computing environment is a client-server environment, in which some of the various functions of the techniques described herein may be performed by a client program product executing on a computer system and some of the functions may be performed by a server program product executing on a server computer. Another example of a distributed computing environment is a cloud computing environment. In a cloud computing environment, computing resources are provided and delivered as a service over a network such as a local-area network (e.g., LAN) or a wide-area network (e.g., the Internet). Examples of cloud-based computing resources may include, without limitation: physical infrastructure resources (e.g., physical computing devices or computer systems, and virtual machines executing thereon) that are allocated on-demand to perform particular tasks and functions; platform infrastructure resources (e.g., an OS, programming language execution environments, database servers, web servers, etc.) that are installed/imaged on-demand onto the allocated physical infrastructure resources; and application software resources (e.g., application servers, single-tenant and multi-tenant software platforms, etc.) that are instantiated and executed on-demand in the environment provided by the platform infrastructure resources. Another example of a distributed computing environment is a computing cluster environment, in which multiple computing devices each with its own OS instance are connected over a fast local network. Another example of a distributed computing environment is a grid computing environment in which multiple, possibly heterogeneous and/or geographically dispersed, computing devices are connected over conventional network(s) to perform a common task or goal. In various distributed computing environments, the information transferred between the various computing devices may be pulled or pushed across the transmission medium that connects the computing devices.

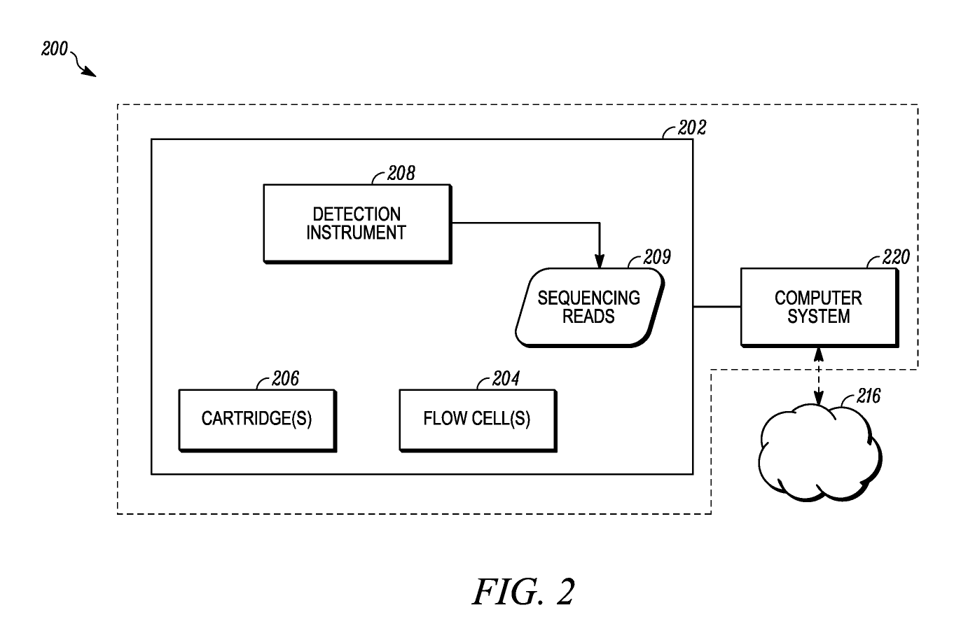

FIG. 2 illustrates an example DNA sequencing system 200 on which some and/or parts of the techniques and methods for multi-round genome processing described herein may be implemented. In some embodiments, DNA sequencing system 200 may be a high throughput instrument capable of sequencing oligos by using any suitable next generation sequencing (NGS) technology. Examples of such DNA sequencing systems include, without limitation, the MiSeq, HiSeq, NextSeq and NovaSeq sequencers manufactured by Illumina, Inc., Ion Proton systems manufactured by Life Technologies, Inc., BGlseq sequencers manufactured by BGI (designed by Complete Genomics, Inc.), or MinION/PromethION sequencers manufactured by Oxford Nanopore Technologies. It is noted, however, that various other DNA sequencing systems available on the market may be suitable for implementing the techniques described herein.

DNA sequencing system 200 includes a sequencing device (sequencer) 202 that is communicatively and/or operatively coupled to computer system 220. Sequencer 202 includes compartments that can accept flow cell(s) or slides 204 with the oligos being sequenced (target oligos), cartridge(s) 206 with the sequencing reagents and buffers used during sequencing, and detection instrument 208 which performs the sequencing. According to the techniques and methods described herein, the target oligos may represent full or partial genomes and/or mixtures thereof. Various fluidic lines, tubing, valves, and other fluidic connections may be used to connect the compartments with flow cell(s) or slides 204 and cartridge(s) 206 to detection instrument 208. A flow cell 204 may include a housing that encloses a solid support (e.g., a microarray, a chip, beads, etc.), with one or more ports being provided for loading the target oligos into the flow cell and for administering the various reagents and buffers during sequencing cycles. In some sequencing systems, the target oligos may be pre-processed into libraries by applying thereto various chemical steps such as denaturing, diluting, etc. A cartridge 206 is used to store various sequencing reagents, buffers, chemicals, as well as any waste that are needed or produced during sequencing. For example, a cartridge 206 may include suitable storage reservoirs that store denaturation agents (e.g., formamide), wash solutions, probes, etc.

Detection instrument 208 is configured to detect the DNA sequences of the target oligos and to generate reads 209. In various embodiments, detection instrument 208 may utilize various sequencing mechanisms such as, for example, sequencing by synthesis, sequencing by ligation, sequencing by hybridization, etc., where such mechanisms may be employed in massively-parallel fashion in order to increase throughput. Further, in various embodiments detection instrument 208 may detect the DNA bases of the target oligos by using optical-based detection, semiconductor-based (or electronic) detection, electrical-based (e.g., nanopore) detection, etc. In various embodiments, detection instrument 208 may also include various suitable mechanical and/or electro-mechanical components that may be configured to position the flow cell 204 at the beginning and/or during sequencing.

Computer system 220 is a suitable computing device and may be communicatively coupled to a network 216. Examples of such computer system and network are described above with respect to FIG. 1. Referring to FIG. 2, computer system 220 is configured to execute software programs that control the operation of sequencer 202 to generate the reads 209 that represent the DNA sequences of the target oligos, in accordance with the techniques described herein. For example, computer system 220 may be configured with suitable software program(s) or application(s) that control the various sequencing cycles performed by sequencer 202. In addition, in some embodiments computer system 220 may be further configured to perform various post-sequencing steps in accordance with the techniques described herein such as, for example, performing error correction on reads 209, assembling longer reads from the generated reads 209, etc.

In operation, computer system 220 controls the operation of DNA sequencing system 200. Sequencing system 200 is first loaded with flow cell(s) or slides 204 that contain the target oligos and with the sequencing cartridge(s) 206. Prior to and/or after loading the flow cells/slides, the target oligos may be amplified (e.g., by using polymerase chain reaction, PCR) in order to preserve a sufficient amount for each read. Then the system performs its sequencing cycles and generates sequencing reads 209 that represent the DNA sequences of the target oligos. A read is generally a sequence of data values that represent (fully or partially) the DNA sequence of a corresponding target oligo. According to the techniques described herein, computer system 220 and the software executing thereon control then perform the methods described herein.

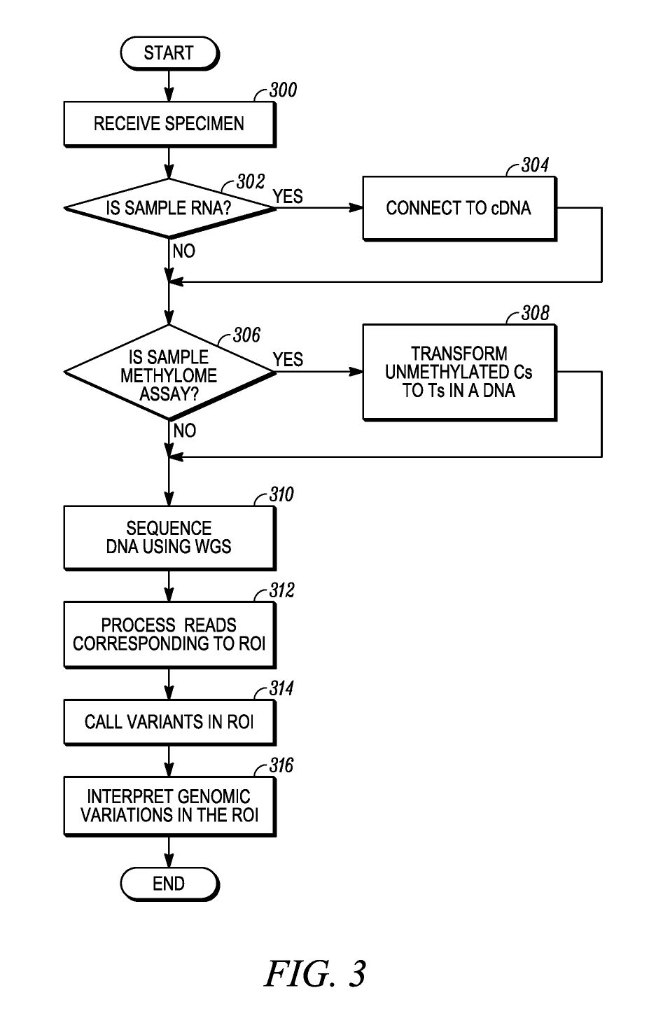

FIG. 3 shows a general testing process for genetic testing in accordance with the principles of the present invention.

In particular, as shown in step 300 of FIG. 3, a nucleic-acid-containing specimen is received (e.g., receive saliva or blood sample from a certain individual). The customer sample may be from an individual. It may also be from a group of individuals. For example, the sample could be the combination of saliva samples from parents and children. This latter mode can identify important (e.g., pathogenic) mutations that may exist in a family, without pointing to the exact individual(s) who carry that trait.

In steps 302, 304, 306 and 308, the nucleic-acid is converted to DNA. For instance, if the sample is RNA (step 302), the RNA is converted to cDNA (step 304). If the sample is a methylome assay (step 306), unmethylated Cs are transformed to Ts in a DNA (step 308). Thus, the nucleic-acid is converted to DNA. If it is DNA to begin with, no conversion is necessary. If it is RNA, a complementary DNA (cDNA) could be made. If it is a methylome assay, busulfite conversion can be used to transform the unmethylated Cs to Ts in a DNA.

In step 310, the resulting DNA is sequenced using whole genome sequencing (WGS), preferably using a PCT-free method to minimize bias caused by errors in the amplification process. Whole genome means there has been no genome reduction/enrichment (such as hybridization methods or amplicon methods) prior to sequencing. Although WGS is the focus of this invention, it must be noted that the methods could be applied to other modalities including exomes and targeted gene panels as well.

The sequenced reads are then saved. For the first customer order, the saved reads are used. The reads that correspond to the specific region of interest (ROI) are selected, i.e., the region that relates to the customer's order. An example of ROI for the first order is the panel of genes that relate to hereditary cancer, e.g., BRCA1, BRCA2. The selection of the reads can be any of the mapping methods that uniquely or semi-uniquely relate the read to the ROI. Examples of such methods are mapping based on alignment or kmer hits. [A kmer is a contiguous or interrupted sequence of k bases.] The kmers utilized in the process could be qualified to be any kmer, or to be only the low-frequency kmers on the reference genome.

As shown in step 312, the reads corresponding to the ROI are processed. The processing could be reference-based, denovo-based or a hybrid of the two methods. It must be noted that the reads that are available at this step may include the reads from that specific ROI or other regions. The latter reads will then have to be suppressed during the process.

Step 314 shows call variants in the ROI.

The genomic variations in the ROI are interpreted in step 316, e.g., to identify pathogenic, likely pathogenic, or other interesting/important variants.

The results may be sent to the interested party or parties, e.g., a customer, customer's physician, etc.)

For all the subsequent customer orders (Order n) (i.e., order #2 and above), the saved reads can be used, with a different region of interest selected based on a new query (ROIn). The ROIn is defined by the selection of test by the customers. For example, a panel of genes that relate to Epilepsy may be selected. The ROIn may be processed using any of the above methods. Call variants in the ROIn are determined, the genomic variations in the ROIn are interpreted, and the results sent to the interested party (e.g., customer, customer's physician, etc.)

The subsequent test can also be done on the same variants discovered in the first test, by applying a new genome interpretation. As the state-of-the-art in interpretation improves (daily, weekly, monthly or annually), the same variants may have different interpretations. In that case, the same variants can be re-run through the interpretation engine to come up with new predictions for the interested parties.

The action of identifying reads for the ROI can be done at the customer's end. In a preferred mode (for security reasons), the customer has the ultimate authority over his/her genome. Then, for each necessary action, e.g., a cancer predisposition test, the customer can use a process that selects reads related to the genes of interest and send them to the genetic test company where the reads would have to be processed, in order to call variants and preferably for the variants to be interpreted and cause a medical decision to be made. This process ensures that the maximum exposure of the customer data to the genetic test company is for the ROI, and therefore potential damages due to exposure are minimized.

After calling variants by the algorithms, the observed variants can be further qualified using a suitable software in-silico verification (ISV) tool that comprises visualizations and textual information related to the sequences. A suitable ISV tool preferably provides visualization of the evidence/support of the raw information (reads) for the called variants. Visualization provided by the ISV tool can be used to identify false positives, by showing anomalous signals corresponding to a variant. ISV visualization can also be used to identify false negatives—by showing signals that look legitimate but have not resulted in variants. In a clinical setting, the ISV visualization plays the role of a safety-net, by giving a human expert the ability to find the truth about the variants, before relying on the effects they may cause per interpretation tool. ISV visualization can be used for all variants. However, since it is time consuming, in a preferred mode, the observed variants may first be passed through the interpretation engine to narrow down the set to what is important. The variants that are verified using ISV visualization can be from one algorithm/pipeline or a set of algorithms/pipelines. For instance, two pipelines can be run on the same data. The discrepancies between the variants (which could be further qualified by pathogenicity of them) may then be resolved using ISV visualization.

In some embodiments, the ROI reads can be used with another sample's complete data or ROI. For instance, the reads from the ROls in a normal and a tumor tissue can be contrasted, either at the read level, or preferably at the variant-identifying signal level or at the final variant level. The reduction of the reads to those of the ROI can be done using the same or different methods, and could be exact or inexact.

### InDel Detection

Reliability of detection can be improved, and false positives can be greatly reduced or even eliminated, by detection of InDels (insertion/deletions). The invention appreciates that the probability of a sequencing error of Sequencing-by-Synthesis (SBS) for InDels is near zero, particularly larger InDels. Importantly, a detected InDel is used to correctly identify an allele, particularly when sequenced with use of a database having low coverage.

In particular, the invention provides high-accuracy DNA sequencing even with low-coverage by defining InDel variation, and regions of InDel variation, and determines a similarity between variations in regions of InDel variation.

Most technologies, e.g., Sequencing-by-Synthesis (SBS), are error-prone in the composition of basecall, and not the position. In other words, the most common error mode is a single base change/error, and not a sequencing error that introduces an insertion, deletion or a combination thereof (collectively called InDels), when reading a sequence. It must be noted that such insertions/deletions (InDels) do not refer to genomic changes as compared to a reference sequence. Rather, these InDels (referred here as “read InDels”) are due to the sequencing errors. In other words, the actual sequence of the molecule is believed to be true. However, the sequencing machine makes an error (in the case of read InDel) that results in the obtained read sequence to appear as if it has an InDel as compared to the actual molecule. In contrast to read InDels, molecule (i.e., “true”) InDels are customarily referred to as InDels. For example, true InDels refer to the cases where the actual molecule that is undergoing sequencing has differences of insertion or deletion type as compared to the reference sequence.

Read InDels have a much lower probability of occurrence, as compared to point mutations, for certain technologies like Illumina's SBS (which is the dominant mode of sequencing in the market). A high accuracy of DNA identification and other low-coverage DNA sequencing is achieved by utilizing true/molecular InDels. Since the read insertions and deletions (read InDels) are not common errors, in case an insertion or a deletion is observed in an SBS (or like) read, what is found may be correlated to the true (InDel) variation. Exceptions to this rule are regions that are known to have high-read-InDel error, e.g., homopolymers of length 10 or higher (which can be excluded in the proposed processes.) Therefore, it can be enabled by a much lower coverage redundancy that is normally required (to recover from the single base errors).

The term Regions of InDel Variability (RIV) is used herein in different contexts. In the most general case RIV includes all the InDels on one's genome. In an alternate context, RIV relates to InDels on certain genes or certain physical locations on the genome. Yet in another context, RIV relates to a predefined set of InDels. RIV can also be defined on a set of InDels, e.g., trinucleotide repeats.

Examples are provided for DNA identification of the genome, in which there are true InDels, with variability across the (e.g., human) population. Examples of such regions are regions associated with InDels with high Minor Allele Frequency (MAF). Other examples include regions with trinucleotide repeats, especially those associated with certain diseases such as Huntington Disease. The human population is known to be highly polymorphic at these sites, and the variations are often in terms of N-base repeats and the number of such repeats. These areas, however, are not limited to trinucleotides for diseases. In fact, most of the long di/tri/quad/penta/hexa-nucleotide variations could be considered for this purpose (as verified by literature search). Multi-base InDels, and even single base InDels, could be used for this application. The less polymorphic the locus, the more loci are needed to achieve the minimum acceptable statistical significance for the purpose of DNA identification. Nevertheless, in general, any InDel that is not in high-error-prone regions (e.g., homopolymers of length 10 or higher, or 15 bases of higher), can be considered for this purpose.

Since the coverage requirement is very low for such purpose, even one read is sufficient for identifying one of the two alleles. If both alleles are required, then a higher number of reads would be required to ensure that both copies are viewed. Even in the latter case, the required coverage is much more relaxed than the coverage required to call bases correctly in the case of single nucleotide variations (SNVs). For instance, a coverage of 10× or 15× is very appropriate for such variation discovery, whereas for a complete genome variation detection, often times 30× or higher coverage is often desired. This is to emphasize that such low coverage (e.g., 10×15×) is not appropriate for single-nucleotide variations (SNV), since that falls into the common mode of error, i.e., single-base error. Read InDels, however, are low probability errors for most technologies (including SBS), and therefore, a method that can discover InDels at low coverage can indeed retain the high accuracy that is needed—e.g., because any such discovered InDels are highly likely to be true InDels (as opposed to read InDels).

### Example 1

Reference:
(SEQ ID NO: 1)
ACGTTTTGACAT
Read bases:
(SEQ ID NO: 2)
ACGTTTTACAT

In the above, the second G is deleted in the read, as compared to the reference. Since this base deletion cannot happen by SBS (with a moderate probability), it is fair to assume (even with a single read), that this deleted base (G) is real—e.g., a true InDel.

### Example 2

Reference:
(SEQ ID NO: 3)
ACGTTTTGACAT
Read bases:
(SEQ ID NO: 4)
ACGTTTTCACAT

Here, the second G in the reference has changed to C that is discovered/detected in the read. Since a single-base change is likely to happen in the SBS process (e.g., because of erroneous base calling), then it is not clear whether this change is a read error or a real point mutation. In order to clarify, one would need to have many reads such as below:

### Example 2a

Reference:
(SEQ ID NO: 5)
ACGTTTTGACAT
Read1 bases:
(SEQ ID NO: 6)
ACGTTTTCACAT
Read2 bases:
(SEQ ID NO: 7)
ACGTTTTCACAT
Read3 bases:
(SEQ ID NO: 8)
ACGTTTTCACAT
Read4 bases:
(SEQ ID NO: 9)
ACGTTTTTACAT
Read5 bases:
(SEQ ID NO: 10)
ACGTTTTCACAT
Read6 bases:
(SEQ ID NO: 11)
ACGTTTTCACAT
Read7 bases:
(SEQ ID NO: 12)
ACGTTTTCACAT
Read8 bases:
(SEQ ID NO: 13)
ACGTTTTCACAT
Read9 bases:
(SEQ ID NO: 14)
ACGTTTTCACAT
Read10 bases:
(SEQ ID NO: 15)
ACGTTTTCACAT
Read30 bases:
(SEQ ID NO: 16)
ACGTTTTCACAT

Here, a large number of reads can point to the fact that the C discovered in the read indeed is real mutation (a true InDel). (Note: an extra, fifth T is an error in Read4.)

### Example 2b

Reference
(SEQ ID NO: 17)
ACGTTTTGACAT
Read1 bases:
(SEQ ID NO: 18)
ACGTTTTCACAT
Read2 bases:
(SEQ ID NO: 19)
ACGTTTTGACAT
Read3 bases:
(SEQ ID NO: 20)
ACGTTTTCACAT
Read4 bases:
(SEQ ID NO: 21)
ACGTTTTTACAT
Read5 bases:
(SEQ ID NO: 22)
ACGTTTTGACAT
Read6 bases:
(SEQ ID NO: 23)
ACGTTTTAACAT
Read7 bases:
(SEQ ID NO: 24)
ACGTTTTGACAT
Read8 bases:
(SEQ ID NO: 25)
ACGTTTTGACAT
Read9 bases:
(SEQ ID NO: 26)
ACGTTTTGACAT
Read10 bases:
(SEQ ID NO: 27)
ACGTTTTGACAT
Read30 bases:
(SEQ ID NO: 28)
ACGTTTTGACAT

Here, the C discovered in read1 in an error. The real base in the actual DNA molecule is still a G, and not a C.

### Example 3

It must be emphasized that the InDels can be of any size (1, 2, and more bases).

Reference:
(SEQ ID NO: 29)
ACGTTTTGTCCACAT
Read bases:
(SEQ ID NO: 30)
ACGTTTTACAT

In the above, a four-base deletion (GTCC) is deleted in the read (in color red), as compared to the reference. Since this base deletion cannot happen by SBS (with a moderate probability), it is fair to assume (even with a single read), that these deleted bases are real, e.g., a true InDel.

### Example 4

Without loss of continuity, we use the term InDel to represent insertions, deletions, block substitutions (and in general non-SNV variations) in genomes. Block substitutions can be thought of a simultaneous deletion and insertion at a certain locus.

Reference:
(SEQ ID NO: 31)
ACGAAAAGTCCACAT
Read bases:
(SEQ ID NO: 32)
ACGTTTTACAT

In the above, the bases at positions 4-11 in the reference are replaced by the bases at positions 4-7 shown in the read. In other words, AAAAGTCC from reference is replaced by TTTT in the read. This represents a Block Substitution, which can be decomposed of a deletion of TTTT in reference followed by the insertion of AAAAGTCC in reference. Once again, since such block substitution cannot happen by SBS (with a moderate probability), it is fair to assume (even with a single read), that these altered bases are real—e.g., a true block substitution (here referred to as InDel).

The statistics of the low coverage InDels works out as follows:
M=300,000 (number of expected InDels in each person)
L=0.1 (a typical low-coverage genome coverage)
G=3 billion (size of the human genome)

P=Probability of a base covered with 1 or more reads˜1−PDFofPoisson(lambda=0.1,x=0)˜0.1 (for L=0.1)

E=Efficiency in the process of sequencing and InDel finding algorithm˜0.4(lack of efficiency)

N=M*P*E=300,000*0.1*0.4−12,000(expected number of InDels in each sample;randomly distributed)

Q=(N/M)*N=N̂2/M=(12000̂2)/3e5˜480(number of InDels that match the same position in any two samples)

MAF=0.075 (worst-case average minor allele frequency for InDels)

S=Q*MAF=36(expected number of the InDels that coincide in any two samples)

Planet=7 billion (population of the planet Earth)

FOM=2̂S/Planet˜10(uniqueness in the whole planet population)

An FOM (figure-of-merit) of 10 is quite strong in making sure no random two individuals would be matched by chance. In other words, for an FOM of 10, 10 times the population of planet (70 billion) should be visited before any two random individuals would be matched by random chance.

It must be noted that even though the FOM is 10 in a typical case (in this example), slight change in efficiency, e.g., from 0.4 to 0.3 can result a drastic loss of this power. For instance, for E=0.3, there would be only 270 matched InDels (Q), which results in S=18.9, which in turn results in FOM of less than 1e-4, which is unacceptable.

Therefore, the sensitivity of such method to the efficiency of the process is quite high. Since the number of InDels in each person is limited, the total power of this method may depend on the InDel algorithm having very high accuracy. In other words, without a high-accuracy InDel calling algorithm, this method may not have the necessary power to be of wide/universal usage (although it may certainly have some limited use and applications).

It is noted that a detected variation (e.g., such as an InDel) may correspond to only one allele. So, by having low coverage, an InDel-based algorithm as described herein will most likely detect at least one InDel or the wildtype. This is fine, since the DNA identification techniques described herein rely on detecting many InDels (˜50%) within a given InDel-variation region, thereby allowing to obtain/detect the copy/allele that actually has the InDel.

It is also noted that an InDel at a given locus may be a two-allele InDel, where one copy/allele may be reference and the other copy/allele may be an InDel (insertion or deletion). In this case, a mechanism may detect only the reference or only the InDel copy, if the overall coverage is low, e.g., 1 copy at that location. However, if the coverage is high enough (e.g., 2, 3 or higher), it is likely that both copies could be detected. In this case, occasionally, both copies may have InDels, which may be similar or different (e.g., of different deletion lengths), or one copy may be deletion and the other copy may be insertion.

It must be noted that the emphasis of this section has been matching two samples that are expected to be from the same source, e.g., matching the DNA in the crime scene (from person A) to a database including M individuals in order to find a match. However, in a general case, the match does not have to be to the same individual, but could be between the person and his/her relatives. For example, in one application of this invention, the DNA profile of a “found” child can be matched to a database of M individuals which includes one or both of the child's parents and not the child's DNA. Assume the database includes the mother of the child. In that case, the match can still be found between the child and the mother. However, the statistical power of the match will be reduced, since the child carries only half of the information content within the Mother's DNA. Without loss of generality, the match can be found between any two relatives (besides parents/children), for example siblings can be matched to each other.

The ability to match an individual to a database that does not include the individual gives this invention a great power. In the case of lost children, the parents can sign up for capturing their DNA profiles in a database after the child is lost. If it was required to match the lost person to a DNA database including that individual, it would defeat the purpose, and the lost person may not be available for DNA profiling.

The application of finding a person using relatives also extends to search for biological parents. In this case, an individual can do his/her DNA profiling, and then assuming one of his/her relatives are captured in a DNA database, the person can find his/her relatives.

Yet another application is in matching individuals with a suitable clinical trial, pharmaceutical company, etc. For instance, an individual may have reason to register themselves, e.g., to make themselves available for candidacy in a current or future clinical trial. Registration ideally covers worldwide clinical trials. A set of users can be signed up and their genomes (or regions thereof) can be sequenced at low-coverage, and saved in a database. Then, an entity (e.g., a pharmaceutical company) who may be interested in a certain study with certain markers can mine the database in order to find potential matches, e.g., individuals having certain InDels in certain regions of interest (ROI). ROI may relate to certain set of InDels. Alternatively, ROI may be defined as the InDels over a set of genes, or a set of physical locations on the genome. These individuals can then be incentivized to provide a higher-coverage DNA sequencing data, perhaps at the expense of the requester. Among the selected ones, a smaller set of individuals will then be selected for participation in the study. The final selection can be done using not only the genomic profile, but also life habits etc. (e.g., provided by a questionnaire).

In the context of low coverage, there are two different possibilities:

1. Super low coverage (SLC). This refers to (for example) 2×, 1× or lower genome coverage. In this mode, basically, it is unlikely to observe both alleles of a locus (for diploid genomes like human). Therefore, it is known that any detected mutation, whether single nucleotide or InDel can only refer to one allele, statistically speaking. This means that with a high probability, the second allele will be missed. The SLC mode is particularly of interest to this invention as that is the only way to achieve very low costs per sample. At the same time, in this mode, since the observations are limited to 1 copy (or a few copies at best), then in the view of the normal mutations (SNV), it would be hard to identify a real mutation from a sequencing error. However, since the emphasis of this invention is on InDels and the sequencing error of SBS is near zero for the InDels, then every detected InDel can be assumed to be true, and therefore can be trusted as the correct identification for one allele.

2. Regular Low Coverage (RLC). This refers to coverages like 3× to 10×, in which there is a reasonable probability of finding both alleles. Since this method is very sensitive, assuming both alleles are observed, a ref/var or var/var scenario would be easy to recognize for var=InDels. This mode is useful where both alleles are needed. For identification purposes, this would not be a hard requirement, although it would increase the power of discrimination. For e-cohort application, this would be more desirable.

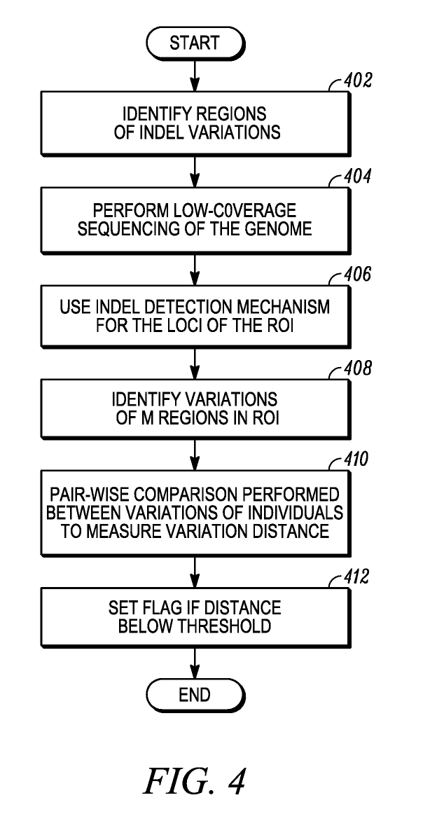

FIG. 4 shows DNA identification based on discovered InDels in low-coverages reads, in accordance with a first embodiment.

In particular, as shown in step 402 of FIG. 4, regions of InDel variations (ROI) that are variable in human population are identified, e.g., some Short Tandem Repeats (often di/tri/quad-nucleotides), or regular InDels. ROI may alternatively be defined as the InDels over a set of genes, or a set of physical locations on the genome.

Step 402 may be made more general, by including all InDels of 2 or more replicates of a pattern, e.g., [CTG]3=CTGCTGCTG (SEQ ID NO:33). Also, one could make it more general by requiring the set to include some of these repeats. Alternatively, any InDel could be used for this purpose.

In step 404, a low-coverage (full genome or selected genome) sequencing of the genome is performed.

Step 404 may be made more general by not limiting it to low-coverage. In fact, the low coverage part could be a dependent claim, e.g., requiring it to be equal to or less than 29×. (Normal genomes are usually sequenced at 30× to 50×, or higher.)

In step 406, an InDel detection mechanism is used for the loci of the ROI.

Step 406 could be made more general by requiring a certain percentage or higher of the loci to be of the type InDel, i.e., not requiring all to be InDels. If the coverage in certain areas are high enough, then SNV alleles that can be called confidently could also be added to the useful loci.

In step 408, variations of at least one copy in M of N (M<=N) regions are identified in the ROI.

Step 408 is just the detection part, so if the loci include more than InDels, it should include SNVs at minimum. The SNVs may have a problem with low coverage. Therefore, if low coverage is used, then SNV becomes less attractive, and InDel becomes the only viable (high-accuracy) modality.

In step 410, a pair-wise comparison is performed between the variations of one individual against a database of K individuals (or P class profiles) to measure the variation distance. This allows for a pattern matching process. Since the InDels are at high accuracy, despite the low coverage a pattern match against a known database is possible, keeping in mind that the database could also be low coverage.

Step 410 is not limited to finding a perfect match. Preferably, this step can be open ended (to the extent various matching algorithms are suitable).

In step 412, a flag is set if the distance is below a predefined threshold.

Step 412 is one way to support Step 410. More generally, instead of a binary flag, which is useful for identification, a different form, perhaps a real number, may be used to be a function of the distance. In fact, Step #6 could be a dependent claim, with the more general step being “performing an action based on the determined variation distance”, with various examples of “actions” being available in various operational contexts.

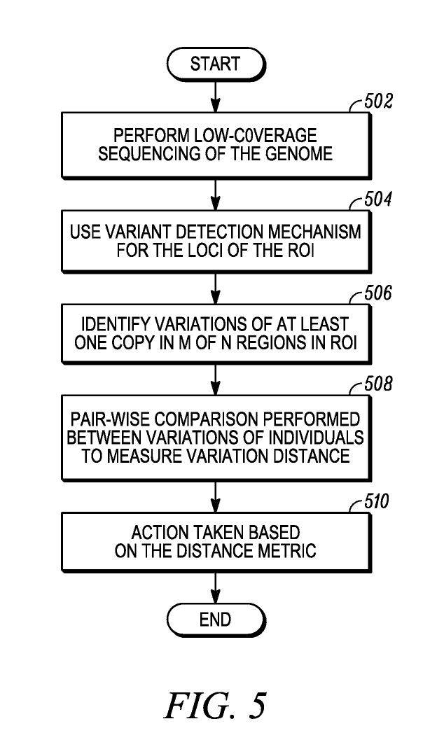

FIG. 5 shows an alternate, more general method of DNA identification based on discovered InDels in low-coverages reads, in accordance with a second embodiment.

In particular, as shown in step 502 of FIG. 5, a low-coverage (full genome or selected genome) sequencing of the genome is performed.

In step 504, a high-accuracy variant detection mechanism is used for the loci of the ROI. These variants could include InDels or SNVs for which enough support is available.

In step 506, variations of at least one copy in M of N (M<=N) regions are identified in the ROI.

In step 508, a pair-wise comparison is performed between the variations of one individual against a database of K individuals (or P class profiles) to measure the variation distance. This allows for a pattern matching process. Since these variants are of high accuracy, despite the low coverage a pattern match against a known database is possible, keeping in mind that the database could also be low coverage.

In step 510, an action is taken based on the distance metric.

### Removing Bias from Genome Testing

One objective of genome testing is to establish whether a person carries a tumor or not. The inventor hereof appreciated that in prior art methods, genome reduction and mapping) may add severe bias to the data, often resulting in many false negatives or false positives. While these steps could also be utilized in this invention, in preferred embodiments otherwise conventional steps are eliminated.

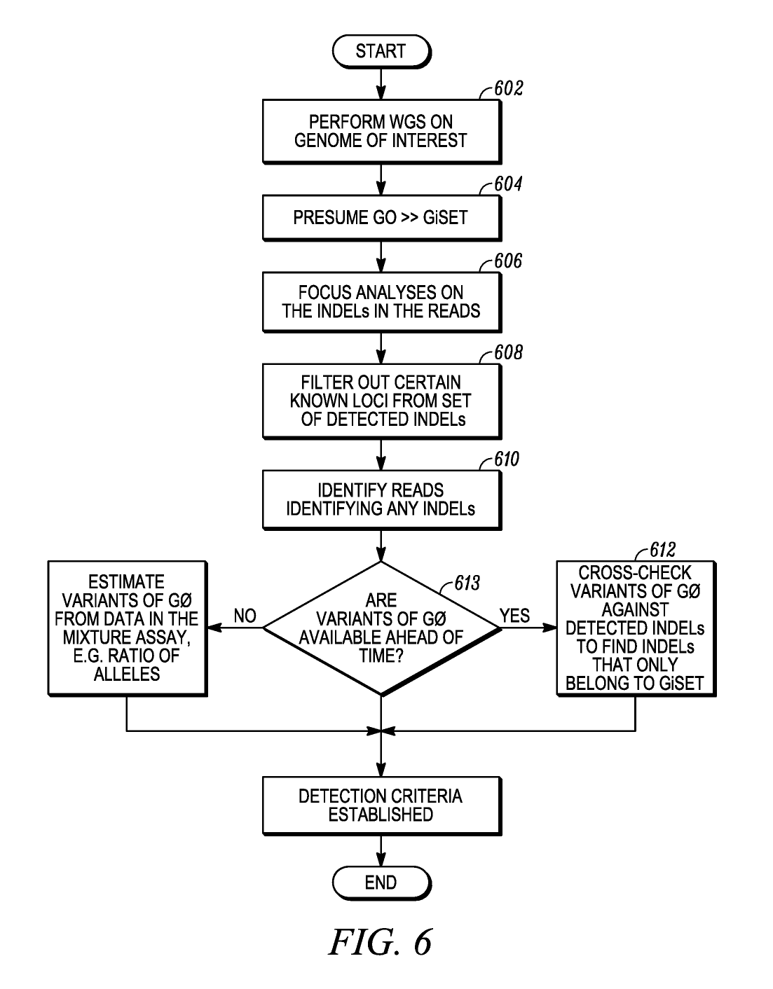

FIG. 6 shows genome testing with bias minimized or removed, in accordance with an embodiment of the present invention.

In particular, as shown in step 602 of FIG. 6, a whole genome sequencing (WGS) operation is performed on the genome of interest. The preferred mode (for having the least bias) for WGS is the PCR-free mode. Also, the while the cost of WGS is high, to minimize the cost WGS could be done at a medium to low coverage, e.g., <50×, <30×, <10× or <1×, which makes the cost manageable. In the preferred mode, to increase the limit of detection (LoD), higher coverages, e.g., >=30×, >=50×, or >=100× could be used.

In step 604, since, in general G0>>GiSet, the concentration of GiSet is miniscule, one would not limit the analyses to a small LOI. Instead, the LOI could be much larger, and potentially as large as the whole genome length or a tangible fraction (e.g., 50% or 90%) of it. For early detection cancer, it can be assumed that GiSet could be as low as 0.01% of the G0 (in contents/concentration). Depending on the cancer, other concentrations may also be fine, e.g., 0.1% or even 1%. Also, if the samples are taken from tumor tissues, higher concentrations can be expected. For liquid biopsy, e.g., in cell-free DNA (cfDNA), lower concentrations can be expected. The expected concentration would be inversely related to the necessary coverage. For instance, cfDNA may require 0.01% detection limit, while for tissue, 0.1% may be sufficient.

In step 606, since, in general, the InDel errors happen at a much lower probability than the single-point mutations (<100 times), the analyses would preferably be focused on the InDels in the reads. This way, once a mutation is found, with a high probability it will belong to either G0 or GiSet, and will not be due to a read error. Let's call the InDels detected in this step the “Detected InDels.” Without loss of generality, the InDels can be used in conjunction with other variant types, like single-nucleotide variants (SNVs).

In step 608, the only exception to the above rule (of having low error for InDels) is certain known loci, e.g., homo-polymers or certain tandem repeats. This is more of a problem for electronic-based sequencing, e.g., in Thermo Fisher's Ion Proton sequencers. However, sequencing-by-synthesis (SBS) methods, such as in Illumina sequencers (e.g., HiSeq) are also not immune to this problem, although the effects are much less in SBS. Nevertheless, such loci are known (or could be learned from previous assays), and therefore can be filtered out from the set of the Detected InDels in the above step. This filtering step can potentially be done prior to finding the InDels to begin with by excluding areas that include known high-InDel-rates.

In step 610, the reads carrying any InDels are identified. This identification could be done via referenced-based mapping, denovo methods, or a combination thereof.

In step 612, if the variants of G0 are available ahead of time (e.g., in an orthogonal assay), they can be cross-checked against the Detected InDels in order to find the InDels that only belong to the GiSet.

In step 614, if the variants of G0 are not available ahead of time, they can be estimated from the data in the mixture assay. This estimation could be done by looking at the ratio of the alleles for the given variant. Depending on the ratio, a homozygous or heterozygous designation could be given to the variations in G0. Denovo or hybrid methods render a better ratio between the two alleles, and therefore would make this step more accurate. Reference-based methods can also be applied. Once the variants of G0 are characterized, they can be cross-checked against the set of detected variants in the mixture, in order to identify the variations (InDels) that only belong to the GiSet.

In step 616, detection criteria may be established by a statistic or a set of statistics on the exclusive/private InDels of the GiSet. For example, in a simple case, the detection criterion could be Number_of_InDels_in_GiSet>Th, where Th is a predefined threshold.

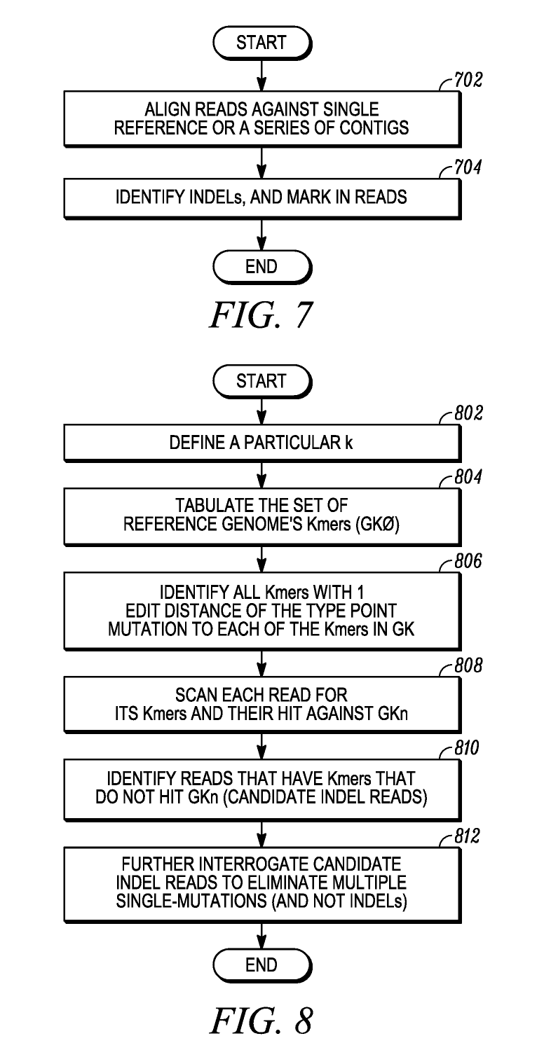

FIG. 7 shows identification of reads containing InDels, in accordance with an embodiment of the present invention.

In particular, as shown in step 702 of FIG. 7, all reads are aligned against a single reference or a series of contigs known as the reference sequence.

In step 704, the alignment identifies the InDels, and such are marked in reads.

FIG. 8 shows an alternative method of identifying the reads with potential InDels, including other mutations.

In particular, as shown in step 802 of FIG. 8, a particular k is defined, e.g., k=21.

In step 804, the set of reference genome's kmers (GK0) is tabulated.

In step 806, all kmers that have 1 edit distance (of the type point mutation) to each of the kmers in GK are identified, and the resultant set is called GK1. The union of the sets GK0 and GK1 is called GK01. The kmers with the edit distance of 2 can also be considered (GK2). However, the set exponentially gets larger, and at some point, it becomes impractical. Nevertheless, if such set is available, the union of that set with the GK01 set is called GK012. For a general case, GKn could denote the union of GKi, where i=0, 1, 2, . . . , n.

In step 808, each read is scanned for its kmers and their hit against GKn. The scanning may be by shifting 1 (most comprehensive scanning) or more bases (less comprehensive scanning). A reasonable trade-off is to shift by k bases.

In step 810, reads that have kmers that do not hit GKn are identified and pulled out for further analysis. We label them as Candidate InDel Reads (CIR). The reads that have all of their scanned kmers hit GKn are believed to have only single-point mutations, and therefore can be discarded for the rest of this analysis, or participate at other parts of the analysis.

In step 812, the CIRs are further interrogated to eliminate the members that have multiple single-mutations (and not InDels).

Definition: When element E is X times unique on the genome it means that there is 1/X probability that E is found on the genome by random chance, assuming that genome is made of random sequences.

Assuming GL=genome length=3e9

uniqueness of GK0=(4̂k)/GL

uniqueness of GK1=(4̂k)/GL/(3*k)

uniqueness of GK01=(4̂k)/GL/(3*k+1)
size of GK0 is GL

size of GK1 is GL*(3*k)

size of GK01 is GL*(3*k+1)
For k=19
GK0 is ˜92 times unique on the genome.
GK1 is ˜1.6 times unique on the genome.
GK01 is ˜1.6 times unique on the genome.
For k=21
GK0 is 1466 times unique on the genome.
GK1 is ˜23 times unique on the genome.
GK01 is ˜23 times unique on the genome.
For k=23
GK0 is ˜23,456 times unique on the genome.
GK1 is ˜340 times unique on the genome.
GK01 is ˜335 times unique on the genome.
For k=25
GK0 is ˜375,300 times unique on the genome.
GK1 is ˜5,004 times unique on the genome.
GK01 is ˜4,938 times unique on the genome.

Assuming the probability of error is fixed for each base, the probability of having a 2-base error on a kmer is as follows:

Even vs. Odd kmers:

While k can be even or odd, an odd kmer is usually preferred as it can distinguish between the top and bottom (a.k.a., forward and reverse) strands of DNA. For an even k, it is possible for the kmer and its reverse complement to be the same, and this results in an ambiguous localization (top vs. bottom strand).

Kmer Size:

On one hand, a longer kmer would result in more uniqueness, which is preferred. This suggests that a kmer longer than 19 is preferred, as a 19mer is only barely unique on the genome.

On the other hand, a longer kmer has a higher probability of having double error hits on the kmer, resulting in a loss of yield. Also, a longer kmer makes the computational problem less tractable, as the number of combinations grow per size of the GKn set.

Computational Complexity:

To make the computations more tractable, a dual-search approach may be implemented by, first, using a shorter kmer, finding a set of candidate reads, and then using the kmer of interest to select the final reads among the candidate reads. This dual-search approach can be generalized to a multi-search approach by using k1, k2, k3, . . . k where k1<k2<k3< . . . <k.

Also, to make the computations more tractable, the reads can first be aligned to the reference genome. Then, those reads that are aligned with only SNV-type mismatches (and not InDels) can be eliminated from the rest of the process.

The below code identifies (for each of the selected genome coverages), the expected number of mixture InDel hits versus the hits from the noise sources (sum of the InDels from the germline origin and read InDels), along with other features.

Based on the below numbers, at the coverage of ˜30 (and above), the mixture hit becomes substantially larger than the noise hit, and therefore, the number of hits can be considered to be from the mixture source. In the below example, a lower bound on the mixture_hit can be found as follows:

std(mixture_hit)˜sqrt(mixture_hit) % based on Poisson Model assumption

mixture_hit−2*std(mixture_hit)

Therefore, for coverage of 30, the threshold would be ˜10, meaning if the number of hits is above 10, one can assume that the effect (sources of GKn, e.g., cancer) exist.

—Parameters Used in the Calculations—
GiSet/G0: 1.0000e-04
efficiency: 0.3000
var0: 300000
var: 63000
base_variant_error: 5.0000e-05
min_variant_length: 2
variant_retention_factor: 0.5000
germline_identification_rate: 0.9900
germline_unidentification_rate: 0.0100
n_alleles: 2
allele_imbalance: 2

—The MATLAB Code—

Below is exemplary Matlab (from MathWorks, Inc.) code for establishing feasibility of this method. Description of some variables and calculations are embedded in the code as comments (starting with %).

v.fish=[1];
% The term “fish” refers to the number of InDel-related molecules that we are

expected to find (fish).

v.coverage=[1 2 10 20 30 40]′;
% Coverage refers to the genome coverage and is a vector, so the calculation

can be done for various coverages.

L = length(v.coverage);
v.genomette_burden=.01/100; % 30 hits
% The term Genomette is used to refer to GiSet. In this case, it is assumed

that the genomette-burden (or in the case of cancer, the tumor-burden is 0.01%). The
0.01% is often a lower bound.

Higher tumor-burdens can be expected, which will result in more favorable

outcomes.

v.efficiency = 0.3; % This refers to the efficiency of the process. For instance,

here it is assumed

that the efficiency is only 30%.
v.var0=300e3; % number of germline variants (InDels)
v.var=126e3/2 ; % number of GiSet-related exclusive variants (InDels)
% This number is based on the assumption that there are 126,000 novel

variants in cancer [cancer genome references]. Also, it is assumed that half of these
variants are InDels.

% v.base_variant_error = (0.5/100)*(10\hat{ }−2);
% It is assumed that the raw base error is 0.5%, and that the InDel error is 2

orders of magnitude (100x) lower than that of the raw base error [DNA sequencing
analysis references].

v.min_variant_length = 2;
% It is assumed that InDels of length 2 and more are considered. In other

words, the InDels of length 1 are deleted from the further processing. This is to reduce
the effect of false read InDels.

switch v.min_variant_length
case 1
v.p_variant_error = v.base_variant_error;
v.variant_retention_factor = 1;
case 2
v.p_variant_error = v.base_variant_error \hat{ } 1.5;
% It is assumed that in the case of InDel of 2 or more, the probability of false

detection is defined as such.

v.variant_retention_factor = 1/v.min_variant_length;
end
% v.coverage_inefficiency = 1/3; % This factor shows how much can the

coverage drop for one of the alleles.

v.germline_identification_rate = 0.99; % full genome variation detection

efficiency. could include the candidate/weak calls.

% v.germline_identification_rate = 0.9; % If dbSNP is used in lieu of the full

genome, this would be the factor that will be used. [This mode is not used in this
particular simulation.]

v.germline_unidentification_rate = 1 − v.germline_identification_rate;
v.n_alleles = 2; % number of alleles in the genome
v.allele_imbalance = 2; % expected or nominal allelic imbalance
t = dataset;
t.coverage = v.coverage;
t.fish = repmat(v.fish,L,1);
t.var = repmat(v.var,L,1);
t.genomette_burden = repmat(v.genomette_burden,L,1);
t.p_variant_error = repmat(v.p_variant_error,L,1);
t.efficiency = repmat(v.efficiency,L,1);
t.germline_identification_rate = repmat(v.germline_identification_rate,L,1);
% t.p_fish1 = binopdf(1,t.coverage,v.genomette_burden);
t.p_fish = 1-binocdf(v.fish-1,t.coverage,v.genomette_burden);
% binopdf and binocdf are the PDF and the CDF of a Binomial distribution,

respectively.

t.false_read_hit = (t.coverage .* v.p_variant_error .* v.var0 .*v.efficiency);
% the false_read_hit represents the number of InDels that are falsely found

(due to the effect of read InDels that are due to errors).

% t.germline_hit= round(
v.germline_unidentification_rate*(binocdf(t.fish,round(v.coverage_inefficiency*

t.coverage),0.5)). *v.var0 );

% germline_hit relates to the number of falsely found InDels that are due to

the germline source.

% germline0 = binopdf(0, round(v.coverage_inefficiency*t.coverage), 0.5);
% germline1minus = binocdf(t.fish,

round(v.coverage_inefficiency*t.coverage), 0.5);

germline_worst_case_coverage = t.coverage/v.n_alleles/v.allele_imbalance;
germline0 = poisspdf(0, germline_worst_case_coverage) / v.n_alleles;
germline1minus = poisscdf(t.fish, germline_worst_case_coverage) /

v.n_alleles;

germline = germline1minus − germline0;
t.germline_hit= ( v.germline_unidentification_rate*(germline).*v.var0 .*

v.efficiency .*v.variant_retention_factor );

t.noise_hit = round(t.false_read_hit + t.germline_hit); % sum of the two source

of false hits t.fish_hit= round( t.p_fish .* v.var .* v.efficiency .* v.variant_retention_factor
);

blim = 2;
fish_lower = t.fish_hit − blim*sqrt(t.fish_hit);
noise_upper = t.noise_hit + blim*sqrt(t.noise_hit);
t.percent_margin =round( 100*(fish_lower − noise_upper)./t.noise_hit );
% the margin shows how separate the two distributions (of real and false hits)

are from each other.

end
----------------------------- End of the MATLAB Code -------------------

### Pooled Sampling

In accordance with embodiments of the invention, samples being tested may be pooled, as disclosed in U.S. Provisional 62/576,075, explicitly incorporated herein by reference. Statistical improvement is obtained with pooled samples among a same family with shared alleles, with immediate family members being stronger than distance family members. Moreover, security and anonymity is inherently obtained with tests of pooled samples, thus avoiding prejudicial use by unauthorized, unintended or other companies.

Population-Level Genetic Testing:

Conventional genetic testing schemes are based on taking a sample from a single patient, performing a test, and repeating it for each additional patient.

A feature of the present invention in accordance with certain embodiments enables an economical method of screening a large population.

A great part of the genetic test is (DNA/RNA/etc.) extraction and library preparation, here collectively called sample preparation. In accordance with the present invention, the overall cost is minimized by reducing the number of sample preparations. The invention is enabled by the fact that many genetic tests look for features (e.g., pathogenic variants) that are extremely rare in populations. For instance, the chance of having hereditary cancer in the general population is believed to be 0.1% to 0.3%. For these arguments, let's assume the frequency is 0.1% or 1 in 1000.

In the prior art, in order to find that 1 patient that has the marker of interest (related to the disease), 1000 patients have to be tested. And, therefore, if the cost of each test is N, the total cost would be 1000*N.

Here, in accordance with an aspect of the invention, samples from a plurality of individuals are pooled into one common sample.

For example, in a first embodiment samples of every two patients are pooled into one combined sample for testing. In such a case, the saliva from 2 patients is combined into one combined saliva. The test is then carried forward with the combined sample. In such an example, the costs for 1000 samples are reduced to the cost of only 1000/2=500 combined samples. The cost of sample preparation for these samples is therefore 500*N (as opposed to 1000*N). When a combined sample includes the affected sample, it will manifest in one of the 500.

It will then have to be resolved to see which sample that specific one is. Therefore, 2 more test are needed. So, overall there would be 500+2=502 sample preparation steps. Hence, the cost of sample preparation has been reduced 1000/502 or almost 2 times. However, it should be noted that the amount of sequencing for the combined sample may need to be more (than that required for 1 sample) in order for the alleles of the affected individual to show up with the same statistical power. In the worst case, the sequencing will have to be twice in depth. However, in practice, a smaller increase might be sufficient.

For the worst case, assuming the cost is composed of sample preparation (N for a sample) and sequencing (S for a depth for a single sample), the cost model would be as follows:

Prior art(without pooling):Total cost=1000*(N+S)

Invention(with pooling of 2):Total cost=502*N+1002*S

So, the cost saving would be:1000*(N+S)−[502*N+1002*S]=488*N−2*S.

Generally, for screening tests 488*N is much larger than 2*S, and therefore, a tangible cost saving would exist.

In practice, the cost saving will be even more as the cost of invention would not quite be double, for instance, it could be 502*N+800*S, and therefore the cost saving would be 488*N+200*S, which is always positive.

Note that the privacy of each individual in the combined sample is inherently protected because of the presence of DNA from two different individuals

The statistical power of pooling gets enhanced if the pooled members belong to the same family, as they share alleles, which would reduce the requirement for doing similar sequencing in the worst case. If the family is immediate family (parent-child), the benefits are maximized. If it includes more distant members, the power of the pooling is still higher that pooling unrelated individuals, but is less than pooling immediate family members.

Pooling can be implemented internally only, particularly if testing for specific signatures. For instance, given 1000 samples to be tested, a portion of the samples may be used for initial pooled testing. Then, if a signature is detected, the samples may then be individually tested again to specifically identify the sample with the signal. In this way the overall number of tests can be reduced, particularly when testing for a rare disease, thus significantly reducing costs.

Pooling can also be implemented externally, meaning that the initial sample received for testing can already be a pooled sample from a plurality of individuals. This ensures anonymity and privacy of each individuals separate DNA.

### Processing of Circulating Tumor Cells (CTCs)

The following embodiments relate to processing of circulating tumor cells (CTCs), and to the definition of patterns including raw coverage curves (RCC), transformed coverage curves (TCC), corrected coverage curves (CCC), or filtered coverage curves (FCC). The following embodiments also relate to differences between ‘normal’ and ‘test’ samples using copy number variation (CNV) including gain or loss of a copy, copy-neutral loss-of-heterozygosity (CnLoH); somatic mutations where the test sample shows a mutation that is absent in the normal (germline) sample; germline mutations that are lost or changed in the test sample; and differences in the context of certain bioinformatics annotations/interpretations.

Variations may be in the form of single-nucleotide variant (SNV), multi-nucleotide variant (MNV), insertion/deletion (InDel), Block Substitution, or structural variation (SV). Some of the following embodiments relate to requiring a significant difference ‘event’, or requiring two or more difference events, preferably in the vicinity of one another. A candidate is identified, and a signature is defined. Normalization may be implemented. Proprietary signatures may be identified.

Whole Genome Sequencing Signatures for Early Detection of Cancer Via Liquid Biopsy:

The invention may be implemented for early detection of cancer using circulating tumor cells (CTCs). While the term early as used herein is primarily used for Stages I and II of cancer, however the invention also lends itself to the later stages of cancer (Stages III and IV), which often forms a simpler problem.

Enthusiasm around early detection of cancer using next-generation sequencing (NGS) has placed this goal in the spotlight, particularly in the recent years. Liquid biopsy is often defined as the modus operandi for early detection, as taking biopsies from the actual organs is not practical for a widespread screening test.

Liquid biopsy comprises cell-free tumor DNA (ctDNA) and circulating tumor cell (CTC) approaches. While ctDNA is more popular, mostly due to the ease of operation, it suffers from low signal-to-noise ratio (SNR). CTC, on the other hand, provides the ability to interrogate single cells with high SNR. However, finding such cells, especially at the earlier stages of cancer, has been challenging.

In addition to the targeted gene panels, whole exome sequencing (WES) and whole genome sequencing (WGS) have been considered in the past, for CTC applications, albeit primarily on prognosis (and not diagnosis). The common ideas hinge upon correlating the count of the CTCs or the discovered copy number variations (CNVs) with the state of the disease or lack thereof.

In this work, our approach has been focused on using WGS for cancer diagnosis, although other NGS modalities may also be considered. We have identified proprietary signatures that have shown promise in identifying cancer versus normal tissues, in specific cancer types such as breast cancer. Some of these signatures have certain properties that would make them portable to the CTC domain.

Since most of publicly available data on CTC work has been on metastatic cancers, we have shown that some signatures hold for such data. Moreover, considering the error modes of CTCs, e.g., allele dropout (ADO), there appears to be a path to maintain the integrity of some of these signatures, although less efficiently, in CTCs from the earlier stages of cancer.

Currently, based on limited data, our approach has shown promise at the WGS tissue level, with a detection rate of ˜90% for Stage I and Stage II of breast cancer. In order to calculate the upper-bound on the sensitivity of this method using liquid biopsy, the tissue-derived number would have to be multiplied by the detection rate of the CTCs, which is currently low to medium, depending on the technology and the cancer type. However, as the CTC detection rate improves, given the R&D efforts in this area, we anticipate that this method would gain more significance in the early detection of cancer.

Methods have been proposed for the processing of circulating tumor cells (CTCs). For instance, Carter et al. “Molecular analysis of circulating tumor cells identifies distinct copy-number profiles in patients with chemosensitive and chemorefractory small-cell lung cancer”, Nature Medicine, 23, 114-119 (2017) performed whole genome sequencing (WGS) of CTC and optionally used a germline sample (as a control), along with a low-coverage sequencing, followed by copy number alteration (CNA) detection.

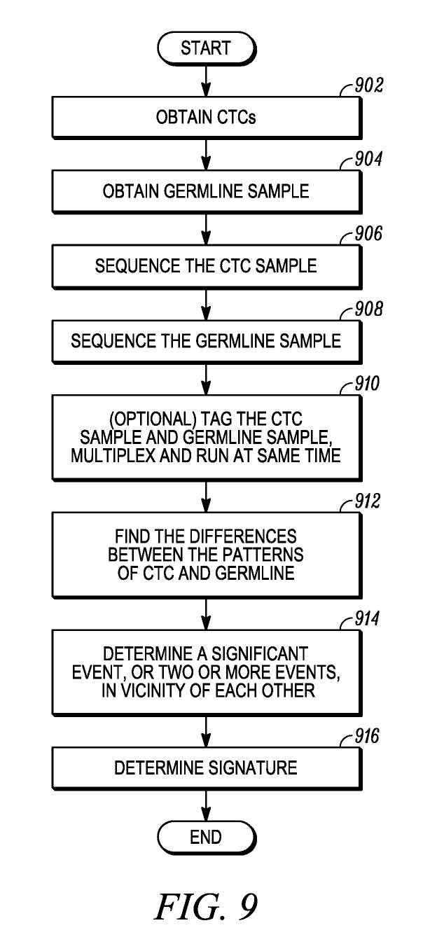

FIG. 9 shows testing of circulating tumor cells (CTCs) for early detection of cancer via liquid biopsy, in accordance with the principles of the present invention.

In particular, as shown in step 902 of FIG. 9, one CTC, a collection of N individual CTCs, a pool of CTCs, or combinations thereof are obtained. The source of CTCs comprises peripheral blood. Commonly, the amounts of 7.5 mL are used for this purpose. However, to increase the odds of catching more CTCs, higher amounts of blood such as 15 mL, 22.5 mL or 30 mL are also possible.

In lieu of 1 CTC, a pool of CTCs can be used.

First, the CTCs are separated from each other and from other cells in the blood, e.g., using DEParray system. The CTCs may be tagged with a unique tag for each CTC. Then, the CTCs are pooled, physically, in order to generate a physical pool of CTCs with low contamination from regular cells. The CTCs may be pooled naturally in the process of enrichment, e.g., through CellSearch System. Then, after processing, some CTCs are pooled informatically, by combining their tags.

In step 904, a Germline sample from the same patient is also obtained. The source of the Germline may be blood or saliva (and other possible sources). For most integrated solution, the same blood that was collected for CTC extraction can be used for the Germline sample.

In step 906, the CTC samples undergo sequencing and the steps that are necessary prior to that, e.g., DNA extraction, amplification and library preparation. The following modes of sequencing are viable: whole genome sequencing (WGS), whole exome sequencing (WES), or targeted gene sequencing (Targeted).

In step 908, the Germline sample undergoes sequencing and the steps that are necessary prior to that, e.g., DNA extraction and library preparation. To minimize biases, the Germline sample should preferably be PCR-free.

Preferably both CTC and Germline are sequenced at a sufficient sequencing depth (e.g., >=5×, >=10×, or >=20×) to allow calls on (preferably) both or at least one allele as well as sensing the difference in copy numbers.

In step 910, optionally, for a balanced run, the CTC and the Germline counterpart can be tagged, multiplexed and run at the same time, to minimize differences due to instrument variations.

While for this embodiment, CTCs and Germline do not have to use the same sequencing modality (e.g., CTC and Germline could be done via WES and WGS, respectively), in a preferred mode of operation, both CTC and Germline would use WGS as the sequencing mode.

In step 912, the patterns of CTC and Germline are compared to find the differences between them.

The patterns could include the raw coverage curves (RCC), transformed (e.g., using a mathematical or look-up operation) coverage curves (TCC), corrected (e.g., corrected for GC-content) coverage curves (CCC), or filtered (e.g., using a low-pass or band-pass digital filter) coverage curves (FCC). The patterns of CTC and Germline could also include variants from each of CTC and Germline. In this context, the differences could be between the variants of CTC, and the Germline calls (variant or reference). Conversely, the differences could be between the variants of Germline, and the CTC calls (variant or reference). The differences could also be between the variants of CTC and the variants of the Germline. A reference call indicates a call where no variants are detected, i.e., the only support at the locus is for the reference base.

The differences may include copy number variation (CNV) including gain or loss of a copy. The loss of copy would result in loss-of-heterozygosity (LoH). The differences could also include copy-neutral loss-of-heterozygosity (CnLoH). (CnLoH cannot be identified using the conventional CNV/CNA methods, as a copy number change is nonexistent for this scenario.) The differences could include somatic mutations where the CTC shows a mutation that is absent in the Germline, i.e., the Germline is reference at that locus. The differences could also include Germline mutations that are lost or changed in the CTC. The differences could relate to the variations in CTC vs. Germline, in the context of certain bioinformatics annotations/interpretations. For instance, it may be a variation in CTC that is marked as pathogenic in ClinVar, whereas this variation is missing from the Germline or is not marked as pathogenic.

In addition to CNV and CnLoH, the variants may be in the form of single-nucleotide variant (SNV), multi-nucleotide variant (MNV), insertion/deletion (InDel), Block Substitution, or structural variation (SV).

In step 914, determine a significant “event,” or two or more “events,” preferably in the vicinity of each other. An “event” is defined by one of the above differences. The definition of the vicinity could be separation by no more than, no less than, or within a certain distance range, e.g., between 1 Kb and 2 Kb. The vicinity may also be defined as all events belong to the same gene, same exon, same intron, or be within a known region, e.g., a 10 Kb region.

In step 916, if N (N=1, 2, 3, . . . ) or more “qualified” events are found in an appropriate vicinity, then the pattern is called a candidate. A series of N or more qualified events is called a Signature. The qualified events, and hence the Signatures, are often cancer-specific.

For instance, for breast cancer, they may be LoH, copy gain, CnLoH, or a combination thereof. The number of qualified events may not only be cancer specific, but also be dependent on the stage of cancer. For instance, for higher stages of cancer, more Signatures may be found. Higher N values provide higher specificity, at the expense of lower sensitivity.

A patient may be declared as being abnormal if one or more expected Signatures are found. The abnormal condition may (by itself or after combining it with other genomic and non-genomic information) be interpreted as the patient having cancer. Otherwise, it will be declared as normal if the support is sufficient but the expected Signatures are not observed. If the support is not sufficient, the status may be called undetermined, suggesting repeated, enhanced, and/or more tests.

To improve the quality, it may be required to have more than one Signature before announcing a patient as having cancer. For instance, Signature 1 may be on having 2 CnLoH events separated by at least 0.1 Kb of each other, on 3 or more genes from a set of known genes on the genome. Signature 2 may be on having 3 or more copy gain events separated by at least 3 Kb from each other on the whole genome.

In a higher-level mode of operation, the above steps can be repeated for each CTC (in Step 902) or combination thereof. Then, a cancer/normal decision may be compiled using the collection of the decisions that are made in each of the Repeats of the steps. For instance, one could require two Repeats, each with a single (and different) CTC, while using the same Germline.

Alternatively, a Signature may be found dynamically using machine learning (ML) (e.g., deep learning) using the “events” (or the constituent elements of the “events”) as the input signals, and the classification (abnormal/cancer vs. normal vs. undetermined) as output. Such ML application may produce the final call or alternatively provide an intermediate call of abnormal. The intermediate call may be combined with other genotypic or phenotypic information to produce the final cancer/normal/undetermined call.

To make sure the variants have enough support, particularly for CTC, it is required to satisfy a validity requirement. This requirement could be a minimum coverage threshold. This minimum coverage threshold may be a specific absolute count on the coverage, e.g., a non-redundant coverage of 5 or more. Non-redundant coverage is the coverage where the repeated reads are collapsed. Alternatively, the minimum coverage threshold may be a specific relative count on the coverage, where the term relative is in relation to the highest coverage point or a certain percentile (e.g., 90th percentile), mean, median, mode, or other values in a certain window (e.g., 10 Kb), a series of windows, or the whole panel, exome, or genome. For example, the relative count threshold could be a number like 0.1 of the mean.

A correct assessment of relative copy number between the CTC and Germline requires a step of normalization. This normalization should be done with the internal signals of each of CTC or Germline. For instance, the Germline signal can be with a coverage of 30× while the CTC signals can be with a coverage of 3×. Therefore, to detect the true differences, these signals must be appropriately normalized, so they are comparable to each other, e.g., with an average of 1 for both, after normalization. The operation of normalization may be explicit (as mentioned above) or implicit (where the downstream process takes into account the differences in the coverages, and does not expect them to be having similar coverages).

In addition to the above signature (Signature 1), the below two signatures can be used for identifying some cancer cases. These signatures for early detection of cancer are as follows:

Signature 2: The use of microstatellite instability (MSI). It is well known that many cancers demonstrate the condition of MSI, as defined by a change in one or both copies of a microsatellite. Microsatellites are the tandem repeats of 2 or more bases. Sometimes homopolymers are also considered microsatellites. If the extracted cancer sample (e.g., from tissue or CTCs) show evidence of microsatellite variation in comparison to the germline variants at the same locus, this event can be marked as a signature of cancer. However, a resilient (to error) signature may require more than 1 event of variation (between somatic and germline). For instance, one could require 3 or more such changes in 1 Mb stretch of genome before classifying the corresponding sample as cancerous. Some examples of MicroSatellite Instability (MSI) for Early Detection of Cancer are as follows:
Example 1: VCF reading in Circulating Tumor Cell (CTC) vs. Normal at a particular locus
CTC: CTCGGGA>ACACGCCTC,ATCGGGA 1/2
Normal: C>T 0/1
Example 2: VCF reading in Tumor (Tissue) vs. Normal at a particular locus
Tumor: T>TTATA,TTATATA 1/2
Normal: No variant (T>T)

Signature 3: Tumor Mutational Burden (TMB). It has been shown that, depending on the cancer type, the number of (somatic) mutations caused by cancer can be large. The number of somatic mutations per 1 Mb is usually defined as the TMB. We measure TMB on the whole genome. Based on the amount of TMB, we will declare the tumor sample (from tissue or CTC) cancerous.

The inventor used real tumor/normal patient data from ICGC. In some colorectal cancer patients, we observed TMB of 10.3, 35.8 and 143.0 mutations/Mb. In some lung cancer patients, we observed TMB of 5.5, 6.7, and 16.4 mutations/Mb. In some glioblastoma patients, we observed 7.3 and 13.3 mutations/Mb. In some breast cancer patients, we observed 0.6, 3.6, 2.4, 6.7, 2.4, 3.0, 1.8, 2.4, and 1.2 mutations/Mb. In some pancreas cancer patients we observed 6.7, 8.5, 9.1, 10.3 and 4.2 mutations/Mb. In some prostate cancer patients, we observed 13.3, 12.1, 4.2, 9.1 and 23.0 mutations/Mb.

This is where for controls (normal vs. normal), the values were mostly 0 or 0.6 mutations/Mb. Therefore, having a threshold of >0.6 would detect most of the above cancers.

The advantage of this invention is the sensitivity to detect in both case (Signatures 2 and/or Signature 3). Based on the inventor's observations, many other genome analysis pipelines in prior art miscall the variants at microsatellites, mostly misclassifying one of the copies as the reference. Consequently, the ability to detect a two-copy change is reduced, significantly. Our genome analysis has sufficient sensitivity and specificity to detect most of these (two-copy) changes, and therefore can use them as a signal for detecting cancer.

### Use of Variants in Normal Genome to Optimize Detection in Test Sample

The ‘normal’ genome variant calls or signals may identify the ‘normal’ existence of such variants for the whole genome or the region of interest (ROI). Then, using the using the variants or primary variant-identifying signals found in the normal genome, the information obtained from the specific test sample may be normalized and optimized.

First, the regular genome variant calls or signals that identify the existence of such ‘normal’ variants for the whole genome or for the regions of interest are determined, using a method that is the most convenient to obtain, while high quality and high quantity (e.g., saliva) and has the most information content—for instance, has low bias (e.g., PCR-free) with relatively long fragment insert sizes (300 to 500 base pairs). Then, use the ‘normal’ variants or the primary variant-identifying signals found in the normal genome to optimize the information obtained from the specific test/application, which is often of a difference source, e.g., blood or tissue. The variant-identifying signals are those that point to the existence of a non-normal variant, e.g., the number of mismatches as compared to the matches, or the number of matches in insertions (or deletions) in the case of an InDel. These signals are markers for either variants or disturbances caused by noise (e.g., in the case of homo-polymers), which may give the appearance of variants but are false positives.

This technique, while providing the necessary information boost, does not have the adverse side-effects of a differential assay. Moreover, there are many advantages. For instance, the acquisition mode for normal may be very cheap and convenient, e.g., saliva. Also, the amount of normal sample may be large for normal variant calling, e.g., saliva. The normal sample acquisition is a one-time event. Also, the information of the normal sample (regular DNA's variants or variant-identifying signals) may be used for any number of tests, as it does not change. Since the normal sample is known to be of a regular diploid genome (in the case of human), i.e. it is known to contain two copies only, the processing of this information is much simpler. Therefore, the quality of the resultant variant calls is higher. Also, since at the time of doing the actual test (for the affected sample), all the variations (in the regions of interest) are known, a non-causal system may be devised to maximally use the normal variant information in the processing of the affected sample. For example, a combination of those normal variants may be considered to enhance the effects caused by the affected sample. Lastly, while the affected sample source may be one with more limited information content, e.g., from a cell-free DNA source (which is normally shorter that a genomic DNA source ˜100-200 bp with the mode of ˜170 bp), the normal samples may enjoy better signal source, e.g., longer DNA fragment/insert sizes (300-500 bp). This longer insert/fragment size highly facilitates the analysis, by increasing the uniqueness of the reads when mapped against the reference genome, or any de novo method.

It is assumed that the genome of interest is from a human (diploid) sample. However, these inventive methods may be applied to other species, in particular those with a ploidy>2, e.g., some plants. Moreover, DNA is used as an exemplary modality for the test. However, it must be noted that many other modalities may be converted to this (DNA-based) modality prior to sequencing.

For instance, RNA may be converted to cDNA, and then the resulting cDNA may be sequenced on DNA sequencing machines. Also, for methylation using bisulfite conversion, the methylation information may be changed to a change (unmethylated C to T) in DNA. Nevertheless, it must be known that even if the test cannot be converted to DNA, in this invention, other signal source modalities may also be considered.

In lieu of mapping (to the reference genome, etc.) one may use a de novo assembly process, where a reference genome is either not used at all or is minimally used.

In all embodiments, the unique mapping to the genome may be replaced with implementation of a de novo assembly.

tDNA could be for one test or for a series of tests. If the latter, these tests may be done in one session or across different times.

In nDNA or tDNA, the term variants may refer to highly-confident variants or lower-confidence ones. These types of variants can collectively be called Candidate Variants and in the abstract form include the variant-identifying signals. The variant-identifying signal may be the signals that show a perturbation of contents as compared to the reference-matching signal. These perturbations may mean the existence of a variant, or may reflect a difficulty of the region. In the former case, the signal may be used directly to discount the effect of the normal in the tumor variants. In the latter case, the region may also be discounted in a similar fashion. However, in this case, the reason is eliminating/reducing noise as opposed to cancelling the effect of the normal. It must be noted that, like variant-identifying signals in the normal data, variant-identifying signals also exist in the tumor sample, and therefore can be contrasted with the signals in the normal to cancel out the effects of the normal. An example of such case is when the detected signals in normal and tumor are paired if they are within an expected short distance from each other, and consequently cancelled, as they would be believed to have come from the same source, i.e., normal variants.

The term affected (such as in affected-normal tests) is used to indicate a potential state, i.e., it means that individual may be affected. Although in this application, the tumor-normal is used as a pair, it must be noted that other pairing possibilities may also exist, for instance tumor from a primary source and tumor from another tissue due to metastasis. Therefore, the concept can be generalized to any type of paired samples.

Also, the concept of pairing, i.e., 2 samples used, can be expanded to include a multiplicity of samples, from instance, one normal and two tumor samples—one from primary tissue and one from metastasis. The concept still holds, as the idea would be to cancel out what is not really coming from a sample (e.g., metastasized) as compared to the variants or signals corresponding to the other samples (e.g., normal and primary tumor).

Consider a test done on an affected sample (with its signal source herein referred to as test DNA or tDNA). Also, assume the normal genome for that specific sample is available and is referred to as the normal DNA (nDNA). In all the below embodiments, it is assumed that the nDNA and tDNA are from the same individual. It is also assumed that the sequencing of the nDNA is preferably done on the whole genome for all applications. However, if the regions of interest are limited, it is possible to apply an enrichment method first and then do the sequencing on the enriched genome. Also, although sequencing nDNA is listed first, for most applications, there is no special order required for sequencing nDNA versus tDNA. In rare cases where the paired tumor and normal samples are not available from the same individual, the pairing (normal and tumor) can happen between the tumor sample of the individual-under-test and the normal sample of a (preferably immediate) family member, or vice versa.

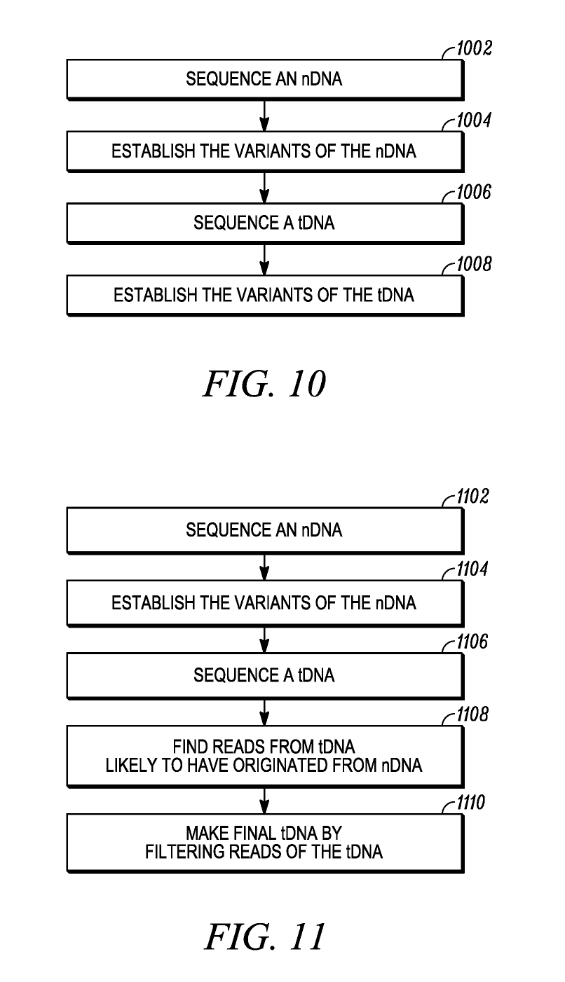

FIG. 10 shows a first exemplary method to contrast variant-identifying signals in a tumor sample, with signals in a normal, to cancel out the effects of the normal.

In particular, in step 1002 of FIG. 10, an nDNA (normal DNA) is sequenced.

In step 1004, the variants and/or variant identifying signals of the nDNA are established.

In step 1006, a tDNA (test DNA) is sequenced.

In step 1008, the variants and/or variant-identifying signals of the tDNA are established, while considering the variants and/or variant-identifying signals of the nDNA. The action of considering/consideration could be done in different ways. For instance, in one exemplary application, the set-difference of the variants of tDNA and nDNA (i.e., what is in tDNA which is not in nDNA) is found and declared as the exclusive/private variants of the tDNA. The difference can also be found at the signal level, by eliminating the signals that happen at the same or nearby positions, between nDNA and tDNA.

FIG. 11 shows a second exemplary method to contrast variant-identifying signals in a tumor sample, with signals in a normal, to cancel out the effects of the normal.

In particular, in step 1102 of FIG. 11, an nDNA is sequenced.

In step 1104, the variants or variant-identifying signals of the nDNA are established.

In step 1106, a tDNA is sequenced.

In step 1108, based on the variants and/or variant-identifying signals of the nDNA, reads from tDNA are found that are likely to have been originated from the nDNA source or not. Depending on the application, a positive or a negative selection of the reads (in terms of matching to the nDNA source) could be passed onto the next stage. Let's refer to these reads as the filtered reads.

In step 1110, the filtered reads of the tDNA (using the variants of the nDNA) are processed in the remaining analysis steps, in order to make the final tDNA calls.

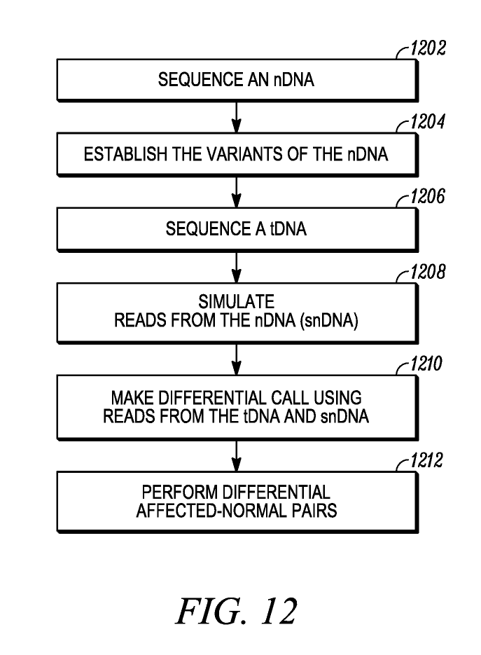

FIG. 12 shows a third exemplary method to contrast variant-identifying signals in a tumor sample, with signals in a normal, to cancel out the effects of the normal.

In particular, as shown in step 1202 of FIG. 12, a normal nDNA is sequenced.

In step 1204, the variants of the nDNA are established.

In step 1206, a tDNA is sequenced.

In step 1208, reads from the nDNA are simulated. Let's call these snDNA reads.

In step 1210, the appropriate differential call is made by using reads from the two sources—tDNA (real reads) and snDNA (synthetic reads).

In step 1212, established otherwise prior art methods of doing a differential affected-normal pairs are performed. The advantage here is that the number of reads and their features (error profiles, etc.) may be tightly matched, as there now is control over the snDNA reads, and it can be made to match the features of the tDNA reads.

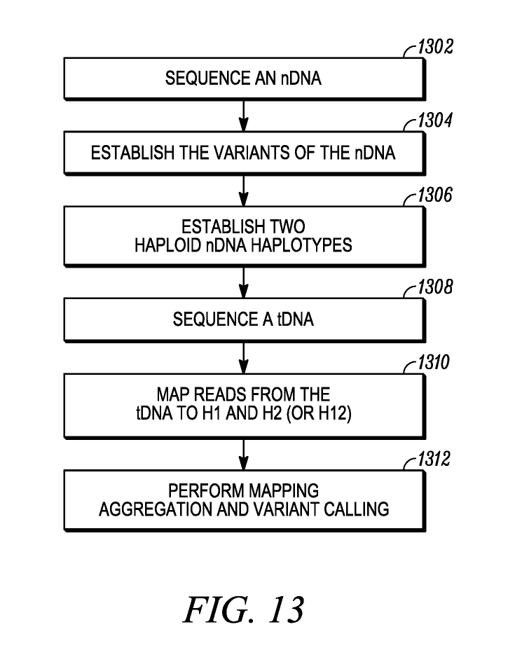

FIG. 13 shows a fourth exemplary method to contrast variant-identifying signals in a tumor sample, with signals in a normal, to cancel out the effects of the normal.

In particular, as shown in step 1302 of FIG. 13, an nDNA is sequenced.

In step 1304, the variants of the nDNA are established.

In step 1306, two haploid nDNA haplotypes (for diploid genomes) are established. This requires phasing information. If the phasing information is not available (such as in most conventional methods), a “piecewise haplotyping” can be performed, where phasing is done for a very short distance, e.g., comparable to the read length, a fraction of that or a few times of that. Let's refer to these as haplotype 1 (H1) and and haplotype 2 (H2). A “piecewise pseudo-haplotyping” could also be done, where the alleles are randomly assigned on the two haplotypes. So long as this is done is a very short distance, e.g., including only one variant, it may work. It is also possible to have a combined H1 and H2 genome (here called H12), where the variants on H1 and H2 are collapsed onto one sequence. This new sequence could be used as a new reference genome, and will not only have regular A/C/G/T/N characters, it will also have polymorphic characters, e.g., S to represent the existence of both C and G at a certain locus. Special characters/strings can be invented to address InDels (insertions/deletions). For instance D_0_3ACT could mean one allele is wildtype/reference (denoted with the digit 0) and the other allele has 3 deletions (ACT).

In step 1308, a tDNA is sequenced.

In step 1310, the reads from the tDNA are mapped to H1 and H2 (or H12) (instead of mapping to the reference genome). The efficacy of the mapping is improved as the H1/H2/H12 do a better representation of the truth for that genome, as opposed to the reference genome. Therefore, the probability of success for uniquely mapping a read increases.

In step 1312, the rest of the processing is performed as in otherwise conventional methods of mapping aggregation and variant calling.

Exemplary tests or applications are now described. The example tests are exemplary only as in most of these applications the information content in the test is limited, and therefore the analysis power will be boosted by using the variant files of the normal DNA.

Exemplary test 1: Methylation assay, using bisulfite conversion. In this case, the genome's alphabet (for most part) is reduced from 4 (A/C/G/T) to 3 (A/C/T). For a sequence of 100 bases, this means a reduction in 3118 billion fold in information content (4̂100/3̂100). Therefore, if a length of 100 was sufficient for uniquely mapping a random read, now this read length is insufficient. For the case of this example, a read length of 126.1 (˜126) is required (4̂100˜3̂126.1) in order to provide a similar statistical power to map a methylome-based read to the genome. Keep in mind that this model was for a random sequence. Knowing that methylation is concentrated in CG-rich areas, the current model may provide a lower bound to the estimated statistical power.

Exemplary test 2: Transcriptome/RNAseq: Often times, single-reads (not pair-ended) are used for transcriptome sequencing. Also, the junctions between the exons in a transcriptome/RNAseq assay poses an important challenge to the transcriptome mapping (as compared to regular genome mapping). The variants on the genome may pose a challenge to the transcriptome mapping as they reduce the probability of success for the mapping of the transcripts to the reference genome. Therefore, by knowing and using the normal DNA's variants, one could account for the expected variations while doing the mapping, and hence improve the probability of successful mapping.

Exemplary test 3: DNA mixture applications: In these tests, a mixture of DNAs exist—often times one of the components is from the genomic/normal DNA sources (G0). This mixture could also include N other sources (G1, G2, G3, . . . Gn). For instance, the N sources could be from N tumor clones. In a majority of cases, the contribution of (i.e., number of reads corresponding to) the nDNA (G0=background) is significantly higher than that of the other sources (G1 . . . GN). Therefore, knowing the variations in the nDNA could simplify the variant calling process, either logically (quality of the calls) or economically (cost of doing the analysis).

Exemplary test 4: Cell-free DNA (cfDNA) applications: In these tests, the cell-free DNA is extracted from the blood, and is subsequently sequenced for finding dissimilarities to the normal person's genome. For instance, one application of cfDNA is in finding tumor-derived variations. Since the length of the cfDNA is often short (between 100 and 240 bases with a mode around 170 bp), mapping it to the reference genome is generally difficult. And, the situation worsens in the regions of the genome with less complexity or in the regions that include repeats. This mapping challenge could cause false positives. In other words, some of the found variants are actually from the germline (nDNA) source, and just because they have had a low mapping efficiency, they can get labeled—falsely—as the tumor-related variants. By knowing, a priori, which variants and/or variant-identifying signals come from the nDNA source, these ambiguous scenarios can be significantly reduced.

Exemplary test 5: DNA from Formalin-Fixed Paraffin-Embedded (FFPE) sources. Similar to the cfDNA, the FFPE-derived DNA (ffpeDNA) could also be short in length (<100 bp). Therefore, mapping uniquely to the genome becomes even harder in these cases. Knowing, a priori, the variants and/or variant-identifying signals of the nDNA can help increase the information content of the ffpeDNA and its success in mapping uniquely to the reference genome.

The above Detailed Description of embodiments is not intended to be exhaustive or to limit the disclosure to the precise form disclosed above. While specific embodiments of, and examples are described above for illustrative purposes, various equivalent modifications are possible within the scope of the system, as those skilled in the art will recognize. For example, while processes or blocks are presented in a given order, alternative embodiments may perform routines having operations, or employ systems having blocks, in a different order, and some processes or blocks may be deleted, moved, added, subdivided, combined, and/or modified. While processes or blocks are at times shown as being performed in series, these processes or blocks may instead be performed in parallel, or may be performed at different times. Further, any specific numbers noted herein are only examples; alternative implementations may employ differing values or ranges.

Unless the context clearly requires otherwise, throughout the description and the claims, References are made herein to routines, subroutines, and modules; generally, it should be understood that a routine is a software program executed by computer hardware and that a subroutine is a software program executed within another routine. However, routines discussed herein may be executed within another routine and subroutines may be executed independently (routines may be subroutines and visa versa). As used herein, the term “module” (or “logic”) may refer to, be part of, or include an Application Specific Integrated Circuit (ASIC), a System on a Chip (SoC), an electronic circuit, a programmed programmable circuit (such as, Field Programmable Gate Array (FPGA)), a processor (shared, dedicated, or group) and/or memory (shared, dedicated, or group) or in another computer hardware component or device that execute one or more software or firmware programs or routines having executable machine instructions (generated from an assembler and/or a compiler) or a combination, a combinational logic circuit, and/or other suitable components with logic that provide the described functionality. Modules may be distinct and independent components integrated by sharing or passing data, or the modules may be subcomponents of a single module, or be split among several modules. The components may be processes running on, or implemented on, a single compute node or distributed among a plurality of compute nodes running in parallel, concurrently, sequentially or a combination, as described more fully in conjunction with the flow diagrams in the figures.

While the invention has been described with reference to the exemplary embodiments thereof, those skilled in the art will be able to make various modifications to the described embodiments of the invention without departing from the true spirit and scope of the invention.

### Using Genome Information in Population-Based Repositories

The main theme of this part invention is to collect genomic information at a reasonable cost and scale the process to a large number of individuals who may be members of one or more social networks. 1) A common mode of such application is doing genome sequencing on each member or each set of members. Sets of members could be immediate relatives, or people with other similar traits, e.g., half siblings, first cousins, second cousins. 2) The genome sequencing comprises different modalities, such as DNA sequencing, RNA sequencing, methylation sequencing, etc. It is assumed that all genome modalities can be converted to DNA prior to sequencing. For instance, in methylation assays, bisulfite conversion can change the methylation state to a different base—unmethylated cytosines are converted to (uracils and consequently) thymines; and then a genome sequencing is done. Also in RNAseq, first a complementary DNA (cDNA) is made from the RNA and then a DNA sequencing is performed. 3) The social network could include one or more set of individuals who are collected in a database, or a series of related databases. Example of such social networks include Facebook and Google+. 4) Smaller social networks such as a particular sports or professional circles could also be considered. Examples of such professional circles are different groups in LinkedIn. 5) The low cost can be achieved by sequencing at a low depth, e.g., 1× coverage. The low cost can also be achieved by methods that are cheaper but are potentially more error-prone, e.g., electronic-based DNA sequencing. 6) In addition to low coverage whole genome, genome-reduced methods (e.g., exome or panels) can be used to reduce cost.

Throughout this invention, it is assumed that a low-cost sequencing and analysis is used to catalogue many people (tens or hundreds of thousand, millions, tens of millions or hundreds of millions). This will enable certain applications such as the ones listed below.

Genealogy: A person's genealogy can be found using certain markers on his/her DNA.

Missing Person: In this application, the genome of a missing person can be matched against the catalogued genome of that individual or those of his/her parents/siblings which may exist in a database. For instance, police may identify a kid in a foreign country who is a suspect victim of child trafficking. In this case, the police may take a sample of saliva from the child, sequence it and then compare it to the main database (social networking) and identify who that person is, directly or indirectly. The direct way would be when the genome of the child is already available in the database (prior to being kidnapped). The indirect way would be when the genome of the suspect victim is matched to the genome of one of his/her parents or siblings. This is when the victim's sample does not exist in the database.

Forensic: In this application, the DNA/RNA/etc. obtained from the physical evidence in a crime scene is sequenced. Such physical evidence could be, for example, the sperm sample from the rapist in a rape scenario. The genome of the criminal is then matched against a database, directly or indirectly. In the direct match, we assume that the criminal's genome is already catalogued in the database, via a previous law enforcement activity. In the indirect way, we assume that the criminal's genome can be matched to one of his parents/siblings/relatives.

Blood Types: Assuming the blood types can be related to loci on the genome, if the person is in need of blood, the circle of his friends can be quickly searched for potential members, assuming the friends' genomes are available but the friends' blood types are not known.

Individual Traits in Matchmaking: When a person is seeking a match on a database, e.g., a dating site, the selection of the candidates can be done not only by phenotypic/social features, e.g., height, weights, color, education, but also using genomic individual features. Such individual features could include those related to phenotypic features (e.g., color of the eyes or certain ancestry background) but also features related to social behavior (e.g., aggressiveness, patience). Of course, the assumption is that such relations have been established. Nevertheless, these relationships do not have to be highly correlative, as partial correlation may be sufficient in ranking the candidates. This is often fine, as the person is often looking for a very small number of candidates (e.g., less than 10) for dating, in a database of potential matches that could include thousands of members.

Pairwise Traits in Matchmaking: This is similar to the above application with the difference that the features are only meaningful when viewed in a pairwise manner, i.e., the genome of the user paired with the genome of any of the candidates. An example of such application is the health of the potential offspring.

Group Traits: These are traits that are common in a group of individuals, e.g., a circle of friends in a social networking platform. Examples of group traits includes being conservative, calm, motivated, and social.

Adoption: A person or couple seeking adoption of a child can benefit from such data. One such example could be searching the database for adoption candidates who are most likely to have features that makes them most similar to the adopted parents, either at the time of adoption or later on in life. These features could include those that are physical, physiological, psychological, or social. Having a common ancestry as to that of the parents or one of the parents is an example.

Sperm/Egg Bank: A person who is interested in using a sperm/egg bank could seek best candidates by selecting samples that are best matched to them. The match of the best could include the donor's physical, physiological, or psychological features. They can also match features that are meaningful in a pairwise manner, for example the probability of the two individuals having a healthy (or as healthy as possible) child, as viewed from the angle of the potential genetic diseases that the child is likely to carry.

Social Networking: A blood relationship can be established in a social networking database using the genomes of a person and those of other people. An example of this is comparing the genome of the individual to his/her friends. By doing so, among the friends of the person, some can be labeled as relatives, and among those the approximate relationship can be established, e.g., parent, sibling, cousin, second cousin, etc. Depending on the type of relationship, different individuals are required. For instance, to establish a relationship as spouse, both individuals and at least one child from them should be available. The circle of friends can be extended to include other members who are not currently joined as friends of the user. This would be the discovery mode, in which the genomes available in the database are scanned in order to find the relevant ones, and make recommendations to the user about the existence of such potential relatives.

Social gaming: A social networking platform could also be a place where online gaming can happen. These games could involve the user's genome in relation to some configurations, other genomes, or combinations thereof. One example of such configuration could be comparing a certain set of loci with some known bases, for lottery purposes. For instance, if the person at Locus 1 has an A, and at Locus 2 has an insertion of GG, then the person wins a prize. All possible mutations as compared to the reference genome can be considered in this case.

In another example, the genome of the person at certain loci is compared to the genome of some other gamers, and if there is a match between any of them, the matching pair can win a prize. The prize could be as simple as getting a chance to meet each other. Of course, the genomic information could be combined with other features, such as proximity and age.

Match2Individuals: The genome of the user(s) (with or without his/her circle of tagged friends) can be scanned for the degree of similarity to the genome of a certain individual, e.g., a celebrity or a group of celebrities. These matchings can be tuned to different genomic markers, perhaps tunable by the user. In a group, the individuals can be ranked based on the degree of similarity (e.g., the count of matches in the mutations) to a particular celebrity, or the aggregate of a group of celebrities. The aggregate can be in the form of intersection, union or other functions done on the mutations derived from the genomes of the group of celebrities. The celebrities are distinguished members, whether they are actors, athletes, academics, etc.

Incidental Findings: When the genome of the person is scanned, some pathogenic mutations may be detected. Such findings can be directly or indirectly (via the user's physician) communicated with the user.

Health Score: Based on the combination of the potential pathogenic or likely pathogenic mutations that are found, a Health Score can be assigned to the individual. This Health Score could be a number between 0 and 100, 100 being the healthiest. The assignment of the Health Score can be done with or without revealing the underlying factors (e.g., genetic variants).

Need4GeneticTest: In this application, the individual's sequenced genome at certain loci gives indications (high likelihood) that the person may be subject to some pathogenic mutation, e.g., certain BRCA1/BRCA2 mutations that could cause cancer. However, the low coverage does not allow having a definitive prediction on such state. In this case, the individual can get an indication from the system that it would be beneficial for him/her to have a specific genetic test (for example breast cancer test) done. This indication can be done in a direct or subtle way. In the direct way, the system lets the user know that there is a slight indication that a pathogenic mutation might be present in his/her genome, and therefore it would be good to consult with the physician. In the subtle way, the system can feed education to the user, e.g., via advertisement, to indicate that the user may be subject to a certain disease or disease class. In summary, this application is a prescreen to the screening or diagnostic testing.

Need4MedicalTest: In this application, the individual's sequenced genome at certain loci gives indications (high likelihood) that the person may be subject to some physiological conditions that are medically disfavored, e.g., high blood pressure. In this case, the individual can get an indication from the system that it would be beneficial for him/her to have a specific medical test (for example cholesterol) done. Therefore, this application improves the health condition of the individuals by referring them to an applicable medical test. And, similar to the previous application, the recommendation can be done in a direct or subtle way.

Tissue Match: When the individual is in need for tissues, the person's MHC region (or other relevant areas) can be compared against a database of genomes that contain similar regions. The idea here is that the bank of tissue types may be more limited in terms of numbers, and a bank of DNA sequences is much more scalable. For instance, one can imagine a database of 100 million sequenced individuals (10% of active members of Facebook), whereas the tissue types are not expected to exceed 1 million, easily. Therefore, such large DNA database, in practice, can replace or complement many existing medical databases.

What is claimed is:

1. A method of performing genetic testing, comprising:
obtaining a first genetic sample from a first person;
obtaining a second genetic sample from a second person;
purposefully mixing at least a portion of the first genetic sample and at least a portion of the second genetic sample into a pooled genetic sample; and
testing the pooled genetic sample for a presence of a signature for a given known ailment.

2. The method of performing genetic testing according to claim 1, further comprising, if the signature is present in the pooled genetic sample:
determining a presence of the signature for the given known ailment from another portion of the first genetic sample; and
determining a presence of the signature for the given known ailment from another portion of the second genetic sample.

3. The method of performing genetic testing according to claim 1, wherein:
the purposefully mixing mixes all of the first genetic sample and all of the second genetic sample into the pooled genetic sample.

4. A method of performing DNA identification using discovered InDels, comprising:
identify at least one region of InDel variation in a genetic sample;
perform low-coverage sequencing of the genome;
detect presence of a first InDel in a loci of the region of InDel variation;
perform a pair-wise comparison of the first InDel to a reference InDel; and
measure a distance between the first InDel and the reference Indel.

5. The method of performing DNA identification using discovered InDels according to claim 4, further comprising:
setting a flag if the distance is below a predetermined threshold.

6. The method of performing DNA identification using discovered InDels according to claim 4, wherein:
the at least one region of InDel variation includes a short tandem repeat.

7. The method of performing DNA identification using discovered InDels according to claim 4, wherein:
the low-coverage sequencing sequences a full genome.

8. The method of performing DNA identification using discovered InDels according to claim 4, wherein:
the low-coverage sequencing sequences a selected sub-portion of the full genome.

9. A method of identifying a read with an InDel mutation in a genetic test, comprising:
identifying a plurality of reference kmers in a reference genome;
identifying a plurality of sample kmers in a test sample;
filtering the plurality of sample kmers to those which have a 1 edit distance from a corresponding one of the plurality of reference kmers;
identifying reads that have kmers that do not have a 1 edit distance from the corresponding one of the plurality of reference kmers; and
eliminating multiple single-mutations from candidate InDel reads.

10. The method of identifying a read with an InDel mutation in a genetic test according to claim 9, further comprising:
filtering the plurality of sample kmers to those which have a 2 edit distance from a corresponding one of the plurality of reference kmers.
